(19) United States
(12) Patent Application Publication
(10) Pub. No.: US 2020/0038299 A1
(43) Pub. Date: Feb. 6, 2020

(54) METHODS AND KITS FOR COLORING HAIR

(71) Applicant: Noxell Corporation, Hunt Valley, MD (US)

(72) Inventors: Elizabeth Hitchcock-Agostino, Loveland, OH (US); Martha Cona, Cincinnati, OH (US); Sebastian Karol Galazka, Loveland, OH (US); Tracy Stephens, New Albany, OH (US); Firoj Vohra, Hackettstown, NJ (US)

(21) Appl. No.: 16/331,921

(22) PCT Filed: Sep. 19, 2017
(86) PCT No.: PCT/US2017/052298

§ 371 Date: Mar. 8, 2019

Related U.S. Application Data
(60) Provisional application No. 62/396,519, filed on Sep. 19, 2016.
(60) Provisional application No. 62/396,529, filed on Sep. 19, 2016.

(51) Int. Cl. A61K 8/22; A61Q 5/10; A61K 8/19

(52) U.S. Cl. CPC A61K 8/22; A61Q 5/10; A61K 2800/884; A61K 2800/4324; A61K 8/19

(57) ABSTRACT

The present invention relates to two methods for coloring hair. The first method foresees the application of a hair coloring composition to the front hair roots, that of a different composition to the back hair roots and optionally that of a third composition to the rest of the hair. The method generates a coloring effect and a masking effect of the new growth which allows a longer lasting coloring effect. The second method foresees the application of a hair coloring composition to the hair roots and that of a different composition to the remaining hair. Also disclosed are kits for performing each of these methods.

### CROSS-REFERENCE TO RELATED APPLICATION

This application claims the benefit of priority to U.S. Provisional Patent Application Ser. No. 62/396,519 entitled “Methods and Kits for Coloring Hair”, filed Sep. 19, 2016, the disclosure of which is incorporated herein in its entirety by reference and to U.S. Provisional Patent Application Ser. No. 62/396,529 entitled “Methods and Kits for Coloring Hair”, filed Sep. 19, 2016, the disclosure of which is incorporated herein in its entirety by reference.

### FIELD OF THE INVENTION

The invention relates to a method of hair coloring, and product kits for performing the method.

### BACKGROUND OF THE INVENTION

Consumers desiring to color their hair typically have two options available namely to use a commercially available retail product or kit or use the services of a professional salon. The latter whilst providing a highly desirable color outcome, is considerably more expensive than the retail option and thus not available to many consumers particularly those who color regularly.

For consumers who have previously colored their hair, the color and condition of the hair is not homogenous along the entire length. The hair strands will comprise root virgin hair or new growth hair which has not been previously colored and conversely at the tips hair which has experienced one or multiple hair coloring treatments. The tips of the hair typically are the most damaged portion of the hair and the color will have changed over time dependant on the wash fade profile and number of types of hair colorant applied, amongst many relevant factors. The intermediate hair length is typically a medium between these two extreme conditions. As a consequence in the salon, for consumers who have previously colored their hair, the salon stylists typically will color the root virgin hair first with a specific composition to most effectively color the hair to the desired end result. However, the remaining length of the hair and tips which has been previously colored and may have undergone multiple coloring cycles is colored with a separate and different composition to that applied at the roots. In this manner the salon stylist aims to provide a homogenous end color result along the entire length of hair from root to tip independent of the variations of starting hair color and condition along the hair length.

However, such a proposition is currently not available to consumers as a retail product, as it would add further to the complexity and cost and is thus not consumer desirable. Nevertheless, experienced home color users continually strive to improve the overall color outcome and indeed as outlined in the instruction packs of many retail products will often direct the consumer to apply the product to the virgin roots first prior to the application of the product along the entire length of hair. However these instructions are imprecise, often not followed, and the results achieved using this method are not comparable with those from a salon stylist. Not only is the overall color result not as homogenous as that provided by a salon stylist, and may result in over deposition of color and over bleaching or brassiness, but the rheology of the product also does not necessarily easily allow specific application at the root line as well as easy pull through the remaining length of hair, whilst ensuring no dripping, and good adhesion to the roots during root application.

In particular, current kits for home coloring of the hair are not fully satisfactory in particular due to insufficient camouflaging of the new growth, especially in consumers with grey/white hair. This means many consumers have to color at high frequency and still have an undesirable root demarcation line after 2-4 weeks.

The method and kits of the present invention helps providing a better camouflage of the new growth so that emerging roots are less noticeable thus allowing the user to recolour the hair less frequently.

### SUMMARY OF THE INVENTION

The present invention relates to a method for coloring hair comprising the steps of
i) mixing a developer component with a dye component to obtain a composition A, wherein the developer component comprises an oxidizing agent and the dye component comprises at least one oxidative dye precursor and an alkalising agent and wherein said composition A comprises 0.1 to 10% of oxidising agent (expressed as grams of hydrogen peroxide in 100 grams of the composition A) and 0.1 to 10% alkalizing agent (expressed as grams of ammonium hydroxide in 100 grams of the composition A);
ii) applying at least a portion of composition A to at least a portion of the front roots of the hair.
iii) provide another composition B said composition B comprising an oxidising agent concentration (expressed as grams of hydrogen peroxide in 100 grams of the composition B) and an alkalizing agent concentration agent (expressed as grams of ammonium hydroxide in 100 grams of the composition B) such that the ratio of concentration of oxidising agent in composition B to concentration of oxidising agent in composition A is 3:4 or lower, and the ratio of the concentration of alkalizing agent in composition B to the concentration of alkalizing agent in composition A is 4:3 or higher,
iv) applying at least a portion of the composition B to at least a portions of the back roots of the hair, vii) Rinsing the hair.
   Optionally in the method of the invention between step iv and vii the following steps are also taken:
v) provide another hair colouring composition C said composition C comprising comprising an oxidizing agent at a concentration wherein the ratio of concentration of oxidizing agent in composition C and the concentration of oxidizing agent in composition A is at least 2:1
vi) applying at least a portion of the composition C to the hair lengths and tips.
   The present invention further relates to a method for coloring hair comprising the steps of
i) mixing a developer component with a dye component to obtain a composition A, wherein the developer component comprises an oxidizing agent and the dye component comprises at least one oxidative dye precursor and an alkalising agent and wherein said composition A comprises 0.1 to 10% of oxidising agent (expressed as grams of hydrogen peroxide in 100 grams of the composition A) and 0.1 to 10% alkalizing agent (expressed as grams of ammonium hydroxide in 100 grams of the composition A);
ii) applying at least a portion of composition A to at least a portion of the roots of the hair,
iii) provide another hair colouring composition C said composition C comprising comprising an oxidizing agent at a concentration wherein the ratio of concentration of oxidizing agent in composition C and the concentration of oxidizing agent in composition A is at least 2:1
iv) applying at least a portion of the composition C to the hair lengths and tips.
v) rinsing the hair

### DETAILED DESCRIPTION OF THE INVENTION

While the specification concludes with claims, which particularly point out and distinctly claim the invention, it is believed the present invention will be better understood from the following description.

As used herein the term “hair” to be treated may be “living” i.e. on a living body or may be “non-living” i.e. in a wig, hairpiece or other aggregation of non-living keratinous fibres. Mammalian, preferably human hair is preferred. However wool, fur and other keratin containing fibres are suitable substrates for the compositions according to the present invention. The terms “root”, “hair roots”, “root hair line” and “virgin hair” all refer to hair which has not been previously treated with a hair coloring composition.

In the preferred embodiment according to the present invention, the hair coloring compositions are applied to hair which has already been previously colored with hair coloring compositions. In such a case, the terms “root”, “hair roots”. “root hair line” and “virgin hair” all refer to the portion of hair having grown, since the last hair coloration, said portion of hair being virgin, i.e. naturally-colored and the terms “hair lengths and tips” refer to the remaining portion of hair having been already previously colored.

In the present application a distinction among “front roots” and “back roots” is introduced. Front and back roots refer respectively to the roots in the front of the head and to the roots in the back of the head. The roots in front of the head are essentially those of the hair encircling the face frame and which are visible by a person looking straight toward the face making eye contact (or in a mirror when making eye contact with the mirrored image). Back roots are those of the hair in the crown and back of the head. The reason for introducing this distinction is that the inventors have found (as it will be explained more in detail below) that in general the front and back roots have different characteristics in term of color and treating them differently generates an overall better effect in term of color uniformity and masking the new growth after coloring. It will be apparent to those skilled in the art that the transition between front roots and back roots is gradual and that front and back roots are not actually separated by a defined line, however for the purpose of the method of the invention we herein define a line of separation between front roots and back roots which is given by the intersection of the head surface with a plane dividing the head in two vertically and which is passing through the ear canal.

All percentages are by weight of the total composition unless specifically stated otherwise. All ratios are weight ratios unless specifically stated otherwise.

### Method for Coloring Hair

The present invention relates to a method for coloring hair as stated herein before.

In step i) of the method a developer component is mixed with a dye component to obtain a composition A. The developer component comprises an oxidizing agent (which may be hydrogen peroxide) and the dye component comprises at least one oxidative dye precursor and an alkalizing agent (which may be ammonium hydroxide). The developer component may comprise from 1 to 20% by weight of the oxidizing agent, the dye component may comprise from 0.01% to 10% by weight of the oxidative dye precursor and/or from 0.1% to 10% by weight of the alkalising agent.

The volume of developer component used in step i) of the method may be from 10 mL to 120 mL, preferably 40 mL to 70 mL, more preferably 55 ml to 65 mL, the volume of dye component used in step i) of the method may be from 10 mL to 120 mL, preferably 15 mL to 70 mL, more preferably 20 mL to 40 mL.

The relative volumes of developer and precursor can vary, for example the relative volumes of developer component and dye component can be from 1:2 to 2:1, but the resulting composition A must have a concentration of oxidizing agent of from 0.1 to 10% (expressed as grams of hydrogen peroxide for 100 grams of composition A) and a concentration of alkalizing agent of from 0.1 to 10% (expressed as grams of ammonium hydroxide per 100 grams of composition A). In some preferred embodiments the concentration of oxidizing agent can be from 0.3 to 6% or from 1 to 3% and, independently, the concentration of alkalizing agent can be from 0.3 to 6% or from 1 to 3%.

The concentration of oxidizing agent and alkalizing agent is expressed as grams of hydrogen peroxide and ammonium hydroxide respectively per 100 grams of the composition. As known to a skilled person in case a different oxidizing agent or alkalizing agent is used the value in grams to consider is obtained by dividing the value in grams of the alternative agent per its equivalent weight and multiplied for the molar weight of the reference agent (hydrogen peroxide or ammonium hydroxide).

The amount of oxidative dye precursor is strongly dependent on the type and shade of dye used and therefore is not an essential feature of the invention. It is within the capacity of a skilled person to determine the correct amount of dye precursor to use based on the result to be obtained. Typical ranges will be discussed below.

The developer component and the dye component may comprise all the customary additives which are typically present in the products of the prior art which will be detailed below. In particular the developer component typically comprises a thickener so that the resulting composition A is viscous enough to be easily applied to the hair using the standard applicators available in industry.

In step ii) of the method at least a portion of the composition A is applied (in non-diluted form) to at least a portion, preferably to more than 50%, more preferably to all the front roots of the hair. Preferably a first portion is applied to the front roots of the hair and a second portion is not applied to the hair (and is set aside for use in step (iii)). The first portion may be from 90 to 10%, preferably from 80 to 20%, more preferably from 70 to 30%, even more preferably from 65 to 35% by weight of the total amount of composition A.

In step iii) of the method a composition B is provided which has a lower concentration of oxidising agent and a higher concentration of alkalizing agent than composition A. More precisely composition B has a concentration of oxidizing agent where the ratio of concentration of oxidising agent in composition B to concentration of oxidising agent in composition A is 3:4 or lower, preferably between 1:10 and 3:4, more preferably between 1:10 and 1:2, even more preferably between 1:10 and 1:3 and the ratio of the concentration of alkalizing agent in composition B to the concentration of alkalizing agent in composition A is 5:4 or higher, preferably between 5:4 and 10:1, more preferably between 3:2 and 7:1, even more preferably between 2:1 and 5:1. Compositions A and B should have the right viscosity to be easily applied using the standard applicators commonly used in hair coloring.

Preferably in step iii) the composition B is provided by mixing the second portion of composition A which was not applied to the hair with a precursor composition D which is essentially oxidising agent free (for essentially “oxidising agent free” it is meant it has less than 1% of oxidising agent, preferably it has no oxidising agent). Such precursor composition D being formulated so that once mixed with the second portion of composition A composition according to the requirements of composition B is obtained.

It is advantageous to obtain the composition B in this way because composition B, may contain dye precursor and oxidising agents in combination and therefore, as known to the skilled persons, these ingredients cannot be stably preserved in a commercial product. In this preferred step the second portion of composition A contains all the oxidising agent which is required in composition B. This second portion of composition A is mixed with the oxidising agent free precursor composition D so to obtain composition B.

Following this process allows to manufacture a Kit for coloring hair wherein compositions A and B can be obtained using a total of 3 bottles (developer component, dye component, precursor composition D).

Alternatively, in a process where composition B is provided independently from composition A, a commercial kit for coloring hair would require 4 bottles to form compositions A and B (developer component and dye component which mixed give composition A+another additional developer component and another additional dye component which mixed give composition B).

Naturally also a method and kit using this 4 bottles approach in obtaining compositions A and B is intended to be within the scope of the present invention, however this method and kit are non-preferred with respect to the 3 bottles approach described above.

In step iv) of the method at least a portion of the composition B is applied (in non-diluted form) to at least a portion, preferably to more than 50%, more preferably to all the back roots of the hair. Preferably a first portion is applied to the back roots of the hair and a second portion is not applied to the hair (and is set aside for use in step (v)). The first portion may be from 90 to 10%, preferably from 80 to 20%, more preferably from 70 to 30%, even more preferably from 65 to 40% by weight of the total amount of composition B.

In step vii) of the method, the hair may be rinsed with water and/or shampoo. After rinsing, they may be further dried and styled as usual. A conditioner component may be applied to the hair after rinsing, preferably prior to drying and styling.

In between step iv) and vii) the remaining portion of the hair (hair lengths and tips) can be optionally treated with an hair coloring solution.

A preferred method is that where in step v) another hair coloring composition C is provided. This composition C comprising an oxidizing agent at a concentration wherein the ratio of concentration of oxidizing agent in composition C and the concentration of oxidizing agent in composition A is at least 2:1, preferably at least 3:1 even more preferably at least 4:1. In step vi) this composition C is applied to the hair lengths and tips, i.e. the remaining portions of the hair which include the portions of the hair which had traces of previous colorations and excludes the new growth.

In a preferred embodiment in step v) the composition C is provided by mixing the second portion of composition B with a precursor composition E said composition E comprising a high level of oxidants and optionally thickeners so that when mixed with the second portion of composition B generates a composition C which has the desired concentration of oxidizing agent and of alkalizing agent (the alkalizing agent mainly coming from the second portion of composition B) and the right viscosity for being applied to the hair with a conventional applicator. Preferably composition C has a low concentration of dyes and/or dye precursors, for example C may have a concentration of dye which is half or less than that of composition A. Typically precursor composition E will be free of dyes and so all the dyes in composition C will come from the second portion of composition B. Alternatively composition C might be free of dyes. This will typically the case where composition C is provided as a pre-prepared composition and not obtained by mixing a precursor composition E with a second portion of composition B.

Preferably composition C has a low concentration of dyes and/or dye precursors, for example C may have a concentration of dye which is half or less than that of composition A. Typically precursor composition E will be free of dyes and so all the dyes in composition C will come from the second portion of composition A. Alternatively composition C might be free of dyes. This will typically the case where composition C is provided as a pre-prepared composition and not obtained by mixing a precursor composition E with a second portion of composition A.

The present invention also encompasses kits for coloring hair following the method described above. In one embodiment in a kit compositions A and C can be obtained using a total of 3 bottles (developer component, dye component, precursor composition E).

Alternatively, in a process where composition C is provided independently from composition A, a commercial kit for coloring hair would require 4 bottles to form compositions A and C (developer component and dye component which mixed give composition A+another additional developer component and another additional dye component which mixed give composition C).

Naturally also a method and kit using this 4 bottles approach in obtaining compositions A and C is intended to be within the scope of the present invention, however this method and kit are non-preferred with respect to the 3 bottles approach described above.

In step v) of the method, the hair may be rinsed with water and/or shampoo. After rinsing, they may be further dried and styled as usual. A conditioner component may be applied to the hair after rinsing, preferably prior to drying and styling.

The advantages provided by this method and kit are that the new growth line (i.e. the demarcation between the new grey growth and previously colored hair) is masked for longer times thus allowing the users to repeat the treatment after a larger amount of time, overall the methods and kits of the invention provide a more vivid and likeable color. The reasons for these effect must be found in the fact that the inventors realized that in general front roots and back roots have a different amount of grey. While the demarcation line may be not the same for all users, it is a general rule that the new roots in the back of the head are darker and may contain less grey/white hair. It has been found that treating the front and back roots with two differently formulated products such as compositions A and B in the present invention leads to better results. In particular with these targeted formulations we are able to reduce the amount of dye to be deposited on the roots vs. traditional product and at the same time obtain a better result, in fact with the optimized conditions (medium bleach low alkalinity at the front and low bleach high alkalinity at the back) we can modify the root line the minimum possible, disguising the grey hair without creating much contrast. Following also the optional step where composition C is applied to the hair lengths and tips the effect is more complete in fact composition C delivers a minimum color deposition on the hair (95% less than traditional compositions) and a high bleaching effect so that it helps removing color buildup from previous coloration and reducing the contrast with the root, so that the hair looks more natural.

In the method of the invention clearly the steps are to be followed in sequence so that the portion of the hair which are treated first will be in contact for a longer time with the composition. The time of application are similar to the time of application of a conventional kit, naturally following the steps in sequence, the time of application of composition A will be longer than the time of application of composition B and the time of application for composition C, when present, will be shorter than both A and B.

The time of application of a composition is measured from when the user terminates the application of that specific composition and when the rinsing of the hair is started. Preferably composition A is applied for a timespan t₁ of from 20 to 60 minutes, composition B is applied for a timespan t₂ which is 2 to 15 minutes shorter than t₁, and when present composition C is applied for a time t₃ which is 2 to 15 minutes shorter than t₂. Typical times for application are 40 minutes for composition A, 35 minutes for composition B and 30 minutes for composition C.

The method of the invention can be easily delivered in kit, prior art kits comprise two containers for the hair coloring components i.e. developer and dye components. In the present kit we will provide in addition the precursor compositions D and optionally E in separate containers. The kit will include instruction to the claimed method in particular instructing the user to obtain composition A by mixing developer and dye components and composition B by mixing a specified residual amount of composition A with precursor composition D and if present to obtain composition C by mixing the residual amount of composition B with precursor composition E. As it will be apparent to those skilled in the art other kits can be provided to perform the method of the invention. For example a kit may contain a developer component, and a dye component which when mixed provide composition A, a second developer and a second dye component which when mixed provide composition B and, optionally, a third developer and a third dye component which when mixed provide composition C.

Alternatively in all kits composition C, not requiring high amount of dye and being possibly even dye free, can be delivered in a kit as a prepackaged composition, so that it is possible to imagine a kit with the following containers:
Developer component,
Dye component
Precursor composition D
Composition C
   or even an alternative kit containing:
Developer component,
Dye component
Second Developer component,
Second Dye component
Composition C
   or even an alternative kit containing:
Developer component,
Dye component
Composition C

The kit may further comprise a shampoo for use in rinsing the hair after coloring and/or a conditioner component.

The kits may also comprise a color refresher component. Such color refresher component may comprise at least one pre-formed dye and may be applied to the hair immediately after the oxidative color. This is typically during the next wash cycle(s) from 1 day to 60 days after the original oxidative application. This color refresher component can be used to increase the initial color obtained and/or boost the color during the wash and style cycle until the next oxidative coloring or bleaching event.

The present invention may be utilized in a variety of packaging and dispensing devices. These dispensing devices can come in the form of separate devices which may be used independently or in combination with one another. Typically, the hair coloring or bleaching components are contained within separate single or multi compartment containers so that the components can be stored separately from one another before use. The components are then mixed together by a mixing means and then applied to the consumer's hair by an application means.

The developer component, the dye component and the precursor compositions may be provided as separate containers in the kit, said container may be a bottle, a tube, an aerosol, or a sachet.

The developer component may be provided as a container such as a bottle, a tube, an aerosol, or a sachet and the dye component may be provided in an additional compartment within the developer container or in a separate container which may be identical such as a dual sachet or aerosol systems for example or different such as a bottle and tube system.

The consumer may mix the developer component and the dye component by any means. This may simply involve the use of a mixing bowl into which the components are dispensed and then mixed, preferably using a mixing means such as a tool. Alternatively, it may involve the addition of one of the components into the container of the other component (typically the dye component is added to the developer component), followed by manual shaking or mixing with a tool. Another system involves the perforation or displacement of a seal located between the separate compartments of the dye component and developer component within a single container or sachet followed by manual mixing within the container or in a separate and/or additional container.

The hair coloring kit may further comprise an applicator. The applicator may be a brush and bowl applicator. Alternatively, the applicator may be a nozzle which may be attached to one of the containers comprised in the kit or a separate applicator device such as a comb or a brush. Such combs and brushes can be adapted in order to achieve particular effects, whether it is quick and even coverage or root/hairline touch up, or highlights or streaks.

Alternatively, one of the containers may be provided with a comb attached to or instead of the dispensing nozzle whereby the product is dispensed through hollow tines and dispensing apertures located in the comb tines. The comb tines may be provided with single or multiple openings along the tines to improve product application and evenness especially root to tip. Product dispensation can be achieved by mechanical pressure applied to the container for example delaminating bottles or any of the mechanisms described hereinabove. The comb may be provided on the container such as to facilitate easy application and may be positioned vertically (so called verticomb) or at an angle to allow the consumer to access all areas.

The components of the kit can be manufactured utilizing any one of the standard approaches, these include a) ‘Oil in water’ process, b) ‘Phase Inversion’ process and c) ‘One-pot’ process. For example, when using “oil in water” process, surfactants of the present invention are added to approximately 50% of total water amount of the compositions at about 90° C., homogenized for 15 to 30 min, then cooled to room temperature to form a premix; this premix is then mixed cold with remaining amounts of water, other optional components and/or oxidizing agent, thus forming the developer component and dye component of the above described coloring kit.

The kit may further comprise a set of instructions comprising instructing the user to color its hair according to the method defined hereinbefore.

The set of instructions may comprise any additional step which is disclosed hereinbefore in the method for coloring hair section of the application.

### Hair Coloring Composition

### Oxidizing Agent

The hair coloring compositions of the present invention comprise a developer component comprising an oxidizing agent. The oxidizing agent is present in an amount sufficient to bleach the melanin pigment in the hair and/or oxidize dye precursors. Typically, such an amount ranges from 1% to 20%, or from 3% to 15%, or from 6% to 12% by weight of the developer component. Inorganic peroxygen materials capable of yielding hydrogen peroxide in an aqueous medium are suitable and include, but are not limited to: hydrogen peroxide; inorganic alkali metal peroxides (e.g. sodium periodate and sodium peroxide); organic peroxides (e.g. urea peroxide, melamine peroxide); inorganic perhydrate salt bleaching compounds (e.g. alkali metal salts of perborates, percarbonates, perphosphates, persilicates, and persulphates, in some embodiments, sodium salts thereof), which may be incorporated as monohydrates, tetrahydrates, etc.; alkali metal bromates; enzymes; and mixtures thereof. Mixtures of two or more oxidizing agents may be used, for example hydrogen peroxide and sodium persulphate. The oxidizing agents may be provided in solution or as a powder which is dissolved prior to use. This is a preferred embodiment for persulphate based oxidizing agents. The oxidizing agent may be hydrogen peroxide. The hair coloring compositions of the invention may comprise air oxidation or auto oxidation hair colorants. In these compositions, the dyes are oxidized by atmospheric oxygen.

### Hair Dyes

The hair coloring compositions of the present invention comprise a dye component comprising at least one oxidative dye precursor and/or an alkalizing agent. Oxidative dye precursors are usually classified either as primary intermediates (also known as developers) or couplers (also known as secondary intermediates). Various couplers may be used with primary intermediates in order to obtain different shades. Oxidative dye precursors may be free bases or the cosmetically acceptable salts thereof.

Typically, the compositions may comprise a total amount of oxidative dye precursors ranging up to 12%, alternatively from 0.1% to 10%, alternatively from 0.3% to about 8%, alternatively from 0.5% to about 6%, by weight of the total composition.

Suitable primary intermediates include, but are not limited to: toluene-2,5-diamine, p-phenylenediamine, N-phenyl-p-phenylenediamine, N,N-bis(2-hydroxyethyl)-p-phenylenediamine, 2-hydroxyethyl-p-phenylenediamine, hydroxypropyl-bis-(N-hydroxyethyl-p-phenylenediamine), 2-methoxymethyl-p-phenylenediamine, 2-(1,2-dihydroxyethyl)-p-phenylenediamine, 2,2′-(2-(4-aminophenylamino)ethylazanediyl)diethanol, 2-(2,5-diamino-4-methoxyphenyl)propane-1,3-diol, 2-(7-amino-2H-benzo[b][1,4]oxazin-4(3H)-yl)ethanol, 2-chloro-p-phenylenediamine, p-aminophenol, p-(methylamino)phenol, 4-amino-m-cresol, 6-amino-m-cresol, 5-ethyl-o-aminophenol, 2-methoxy-p-phenylenediamine, 2,2′-methylenebis-4-aminophenol, 2,4,5,6-tetraminopyrimidine, 2,5,6-triamino-4-pyrimidinol, 1-hydroxyethyl-4,5-diaminopyrazole sulfate, 4,5-diamino-1-methylpyrazole, 4,5-diamino-1-ethylpyrazole, 4,5-diamino-1-isopropylpyrazole, 4,5-diamino-1-butylpyrazole, 4,5-diamino-1-pentylpyrazole, 4,5-diamino-1-benzylpyrazole, 2,3-diamino-6,7-dihydropyrazolo[1,2-a]pyrazol-1 (5H)-one dimethosulfonate, 4,5-diamino-1-hexylpyrazole, 4,5-diamino-1-heptylpyrazole, methoxymethyl-1,4-diaminobenzene, N,N-bis(2-hydroxyethyl)-N-(4-aminophenyl)-1,2-diaminoethane, salts thereof and mixtures thereof.

Suitable couplers include, but are not limited to: resorcinol, 4-chlororesorcinol, 2-chlororesorcinol, 2-methylresorcinol, 4,6-dichlorobenzene-1,3-diol, 2,4-dimethylbenzene-1,3-diol, m-aminophenol, 4-amino-2-hydroxytoluene, 2-methyl-5-hydroxyethylaminophenol, 3-amino-2,6-dimethylphenol, 3-amino-2,4-dichlorophenol, 5-amino-6-chloro-o-cresol, 5-amino-4-chloro-o-cresol, 6-hydroxybenzomorpholine, 2-amino-5-ethylphenol, 2-amino-5-phenylphenol, 2-amino-5-methylphenol, 2-amino-6-methylphenol, 2-amino-5-ethoxyphenol, 5-methyl-2-(methylamino)phenol, 2,4-diaminophenoxyethanol, 2-amino-4-hydroxyethylaminoanisole, 1,3-bis-(2,4-diaminophenoxy)-propane, 2,2′-(2-methyl-1,3-phenylene)bis(azanediyl)diethanol, benzene-1,3-diamine, 2,2′-(4,6-diamino-1,3-phenylene)bis(oxy)diethanol, 3-(pyrrolidin-1-yl)aniline, 1-(3-(dimethylamino)phenyl)urea, 1-(3-aminophenyl)urea, 1-naphthol, 2-methyl-1-naphthol, 1,5-naphthalenediol, 2,7-naphthalenediol or 1-acetoxy-2-methylnaphthalene, 4-chloro-2-methylnaphthalen-1-ol, 4-methoxy-2-methylnaphthalen-1-ol, 2,6-dihydroxy-3,4-dimethylpyridine, 2,6-dimethoxy-3,5-pyridinediamine, 3-amino-2-methylamino-6-methoxypyridine, 2-amino-3-hydroxypyridine, 2,6-diaminopyridine, pyridine-2,6-diol, 5,6-dihydroxyindole, 6-hydroxyindole, 5,6-dihydroxyindoline, 3-methyl-1-phenyl-1H-pyrazol-5(4H)-one, 1,2,4-trihydroxybenzene, 2-(benzo[d][1,3]dioxol-5-ylamino)ethanol (also known as hydroxyethyl-3,4-methylenedioxyaniline), and mixtures thereof.

The primary intermediates and couplers are usually incorporated into the dye component.

### Alkalizing Agent

The dye component of the hair coloring compositions may optionally comprise an alkalizing agent, preferably a source of ammonium ions and/or ammonia. Alkalizing agent may include alkanolamines, for example monoethanolamine, diethanolamine, triethanolamine, monopropanolamine, dipropanolamine, tripropanolamine, 2-amino-2-methyl-1,3-propanediol, 2-amino-2-methyl-1-propanol, and 2-amino-2-hydroxymethyl-1,3-propanediol; guanidium salts; and alkali-metal and ammonium hydroxides and carbonates, such as sodium hydroxide and ammonium carbonate; and mixtures thereof. A preferred alkalizing agent is monoethanolamine. Particularly, preferred alkalizing agents are those which provide a source of ammonium ions. Any source of ammonium ions is suitable for use herein. Preferred sources include ammonium chloride, ammonium sulphate, ammonium nitrate, ammonium phosphate, ammonium acetate, ammonium carbonate, ammonium hydrogen carbonate, ammonium carbamate, ammonium hydroxide, ammonium percarbonate salts, ammonia and mixtures thereof. Particularly preferred are ammonium carbonate, ammonium carbamate, ammonia, ammonium hydroxide and mixtures thereof. The hair coloring compositions of the present invention may comprise from 0.1% to 10%, or from 0.5% to 5%, or from 1% to 3% by weight of an alkalizing agent, preferably ammonium ions. Preferably, if present, the ammonium ions and carbonate ions are present in the hair coloring composition at a weight ratio of from 3:1 to 1:10, preferably 2:1 to 1:5.

The dye component may comprise from 0.1% to 5%, or from 0.2% to 3%, or from 0.5% to 2% by weight of an alkalizing agent.

### Composition Components

Compositions A, B C and precursor compositions D and E may further comprise additional components including but not limited to: solvents; direct dyes; radical scavengers; thickeners and or rheology modifiers; chelants; pH modifiers and buffering agents; carbonate ion sources; peroxymonocarbonate ion sources; surfactants; polymers; fragrances; enzymes; dispersing agents; peroxide stabilizing agents; antioxidants; natural ingredients, e.g. proteins and protein derivatives, and plant extracts; conditioning agents; ceramides, preserving agents; and opacifiers and pearling agents (such as titanium dioxide and mica). These components may be comprised in the developer component, the dye component, or in the precursor compositions. Some adjuvants referred to above, but not specifically described below, which are suitable are listed in the International Cosmetics Ingredient Dictionary and Handbook, (8th ed.; The Cosmetics, Toiletry, and Fragrance Association). Particularly, vol. 2, sections 3 (Chemical Classes) and 4 (Functions) are useful in identifying specific adjuvants to achieve a particular purpose or multipurpose.

### Direct Dyes

The hair coloring compositions may comprise compatible direct dyes in an amount sufficient to provide coloring, particularly with regard to intensity. Typically, such an amount will range from 0.05% to 4%, by weight of the hair coloring composition. Suitable direct dyes include but are not limited to: Acid Yellow 1; Acid Orange 3; Disperse Red 17; Basic Brown 17; Acid Black 52; Acid Black 1; Disperse Violet 4; 4-nitro-o-phenylenediamine; 2-nitro-p-phenylenediamine; Picramic Acid; HC Red No. 13; 1,4-bis-(2′-hydroxyethyl)-amino-2-nitrobenzene; HC Yellow No. 5; HC Red No. 7; HC Blue No. 2; HC Yellow No. 4; HC Yellow No. 2; HC Orange No. 1; HC Red No. 1; 2-chloro-5-nitro-N-hydroxyethyl-p-phenylenediamine; HC Red No. 3; 4-amino-3-nitrophenol; 2-hydroxyethylamino-5-nitroanisole; 3-nitro-p-hydroxyethylaminophenol; 2-amino-3-nitrophenol; 6-nitro-o-toluidine; 3-methylamino-4-nitrophenoxyethanol; 2-nitro-5-glycerylmethylaniline; HC Yellow No. II; HC Violet No. 1; HC Orange No. 2; HC Orange No. 3; HC Yellow No. 9; 4-nitrophenyl aminoethylurea; HC Red No. 10; HC Red No. 11; 2-hydroxyethyl picramic acid HC Blue No. 12; HC Yellow No. 6; hydroxyethyl-2-nitro-p-toluidine; HC Yellow No. 12; HC Blue No. 10; HC Yellow No. 7; HC Yellow No. 10; HC Blue No. 9; N-ethyl-3-nitro PABA; 4-amino-2-nitrophenyl-amine-2′-carboxylic acid; 2-chloro-6-ethylamino-4-nitrophenol; 6-nitro-2,5-pyridinediamine; HC Violet No. 2; 2-amino-6-chloro-4-nitrophenol; 4-hydroxypropylamino-3-nitrophenol; HC Yellow No. 13; 1,2,3,4-tetrahydro-6-nitrochinoxalin; HC Red No. 14; HC Yellow No. 15; HC Yellow No. 14; 3-amino-6-methylamino-2-nitropyridine; 2,6-diamino-3-((pyridine-3-yl)azo)pyridine; Basic Red No. 118; Basic Orange No. 69; N-(2-nitro-4-aminophenyl)-allylamine; 4-[(4-amino-3-methylphenyl)(4-imino-3-methyl-2,5-cyclohexadien-1-ylidene) methyl]-2-methyl-benzeneamine-hydrochloride; 2-[[4-(dimethyl-amino)phenyl]azo]-1,3-dimethyl-1H-imidazolium chloride; 1-methyl-4-[(methylphenyl-hydrazono)methyl]-pyridinium, methyl sulfate; 2-[(4-aminophenyl)azo]-1,3-dimethyl-1H-imidazolium chloride; Basic Red 22; Basic Red 76; Basic Brown 16; Basic Yellow 57; 7-(2′,4′-dimethyl-5′-sulfophenylazo)-5-sulfo-8-hydroxynaphthalene; Acid Orange 7; Acid Red 33; 1-(3′-nitro-5′-sulfo-6′-oxophenylazo)-oxo-naphthalene chromium complex; Acid Yellow 23; Acid Blue 9; Basic Violet 14; Basic Blue 7; Basic Blue 26; sodium salt of mixture of mono- & disulfonic acids (mainly the latter) of quinophthlanone or 2-quinolylindandione; Basic Red 2; Basic Blue 99; Disperse Red 15; Acid Violet 43; Disperse Violet 1; Acid Blue 62; Pigment Blue 15; Acid Black 132; Basic Yellow 29; Disperse Black 9; 1-(N-methylmorpholinium-propylamino)-4-hydroxy-anthraquinone methylsulfate; HC Blue No. 8; HC Red No. 8; HC Green No. 1; HC Red No. 9; 2-hydroxy-1,4-naphthoquinone; Acid Blue 199; Acid Blue 25; Acid Red 4; Henna Red; Indigo; Cochenille; HC Blue No. 14; Disperse Blue 23; Disperse Blue 3; Disperse Blue 377; Basic Red 51; Basic Orange 31; Basic Yellow 87; and mixtures thereof. Preferred direct dyes include but are not limited to: Disperse Black 9; HC Yellow 2; HC Yellow 4; HC Yellow 15; 4-nitro-o-phenylenediamine; 2-amino-6-chloro-4-nitrophenol; HC Red 3; Disperse Violet 1; HC Blue 2; Disperse Blue 3; Disperse Blue 377; Basic Red 51; Basic Orange 31; Basic Yellow 87; and mixtures thereof.

### pH Modifiers and Buffering Agents

The hair coloring compositions of the present invention may have a pH of from 12 to 7.5, or from 11 to 8.4, or from 10 to 8.5. They may further comprise a pH modifier and/or buffering agent in an amount that is sufficiently effective to adjust the pH of the composition to fall within a range from 3 to 13, or from 7.5 to 12, or from 8.5 to 9.5 in some embodiments particularly those comprising a source of carbonate ions. Preferably the pH modifier or buffering agent is comprised in the dye component.

Suitable pH modifiers and/or buffering agents for use herein include, but are not limited to: ammonia, alkanolamides such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3,-propandiol and guanidium salts, alkali metal and ammonium hydroxides and carbonates; in some embodiments, sodium hydroxide and ammonium carbonate, and acidulents such as inorganic and inorganic acids, e.g., phosphoric acid, acetic acid, ascorbic acid, citric acid or tartaric acid, hydrochloric acid, and mixtures thereof.

### Chelants

The hair coloring compositions of the present invention may contain a chelant (or “chelating agent” or “sequestering agent” or “sequestrant”) or salt thereof. The presence of redox metals such as copper, iron, and calcium in tap water used by consumers, though only present at low levels of 5 to 100 ppm and, for calcium, 4000 to 10000 ppm, may effect the color chemistry of oxidative hair dyes. The presence of chelants may limit this effect. The term “salts thereof” means all salts comprising the same functional structure as the chelant they are referring to and includes alkali metal, alkaline earth, ammonium, substituted ammonium salts (e.g., monoethanolammonium, diethanolammonium, triethanolammonium), in particular all sodium, potassium or ammonium salts. The hair coloring compositions may comprise from 0.01% to 5%, or from 0.25% to 3%, or from 0.5% to 1% by weight of chelant, salts thereof, derivatives thereof, or mixtures thereof. The chelant may be present in any component.

Chelants are well known in the art and a non-exhaustive list thereof can be found in A E Martell & R M Smith, Critical Stability Constants, Vol. I, Plenum Press, New York & London (1974) and AE Martell & RD Hancock, Metal Complexes in Aqueous Solution, Plenum Press, New York & London (1996), both incorporated herein by reference. Suitable chelants for use herein are carboxylic acids (in particular aminocarboxylic acids), phosphonic acids (in particular aminophosphonic acids) and polyphosphoric acids (e.g., linear polyphosphoric acids), and the salts thereof.

Aminocarboxylic acid chelants for use herein have at least one carboxylic acid moiety (—COOH) and at least one nitrogen atom. Examples of aminocarboxylic acid chelants suitable for use herein include diethylenetriamine pentaacetic acid (DTPA), ethylenediamine disuccinic acid (EDDS), ethylenediamine diglutaric acid (EDGA), 2-hydroxypropylenediamine disuccinic acid (HPDS), glycinamide-N,N′-disuccinic acid (GADS), ethylenediamine-N—N′-diglutaric acid (EDDG), 2-hydroxypropylenediamine-N—N′-disuccinic acid (HPDDS), ethylenediaminetetraacetic acid (EDTA), ethylenedicysteic acid (EDC), EDDHA (ethylenediamine-N—N′-bis(ortho-hydroxyphenyl acetic acid)), diaminoalkyldi(sulfosuccinic acids) (DDS), N,N′-bis(2-hydroxybenzyl)ethylenediamine-N,N′-diacetic acid (HBED) and salts thereof and mixtures thereof.

Other suitable aminocarboxylic type chelants for use herein are iminodiacetic acid derivatives such as N-2-hydroxyethyl N,N diacetic acid or glyceryl imino diacetic acid, iminodiacetic acid-N-2-hydroxypropyl sulfonic acid and aspartic acid N-carboxymethyl N-2-hydroxypropyl-3-sulfonic acid, 0-alanine-N,N′-diacetic acid, aspartic acid-N,N′-diacetic acid, aspartic acid-N-monoacetic acid and iminodisuccinic acid chelants, ethanoldiglycine acid, and salts thereof and mixtures thereof. Dipicolinic acid and 2-phosphonobulane-1,2,4-tricarboxylic acid are also suitable. Preferred for use herein is ethylenediamine-N,N′-disuccinic acid (EDDS), and salts thereof.

Amino-phosphonic acid type chelants suitable for use herein are defined as chelants comprising an amino-phosphonic acid moiety (—PO₃H₂) or its derivative —PO₃R₂ wherein R₂ is a C₁ to C₆ alkyl or aryl radical and salts thereof.

Suitable amino-phosphonic acid type chelants for use herein are aminotri-(1-ethylphosphonic acid), ethylenediaminetetra-(1-ethylphosphonic acid), aminotri-(1-propylphosphonic acid), and aminotri-(isopropylphosphonic acid). Preferred chelants for use herein are aminotri-(methylenephosphonic acid), ethylene-diamine-tetra-(methylenephosphonic acid) (EDTMP) and diethylene-triamine-penta-(methylenephosphonic acid) (DTPMP) and mixtures thereof.

Examples of other chelants suitable for use herein include but are not limited to polyethyleneimines, polyphosphoric acid chelants, etidronic acid, Methylglycine diacetic acid N-(2-hydroxyethyl)iminodiacetic acid, Iminodisuccinnic acid, N,N-Dicarboxymethyl-L-glutamic acid and N-lauroyl-N,N,N′-ethylenediamine diacetic acid.

In some embodiments, the hair coloring compositions of the invention comprise a carboxylic acid chelant, a phosphonic acid chelant, a polyphosphoric acid chelant, salts thereof, or mixtures thereof. In certain embodiments, the compositions of the invention comprise diethylenetriamine pentaacetic acid (DTPA), ethylenediamine-N,N′-disuccinic acid (EDDS), ethylenediamine-N,N′-diglutaric acid (EDDG), 2-hydroxypropylenediamine-N,N′-disuccinic acid (HPDDS), glycinamide-N,N′-disuccinic acid (GADS), ethylenediamine-N—N′-bis(ortho-hydroxyphenyl acetic acid) (EDDHA), diethylene-triamine-penta-(methylenephosphonic acid) (DTPMP), salts thereof, or mixtures thereof. In further embodiments, the compositions of the invention comprise from 0.1% to 5% of diethylene-triamine-penta-(methylenephosphonic acid) and from 0.1% to 5% of ethylenediamine-N,N′-disuccinic acid.

### Radical Scavenger

The hair coloring compositions according to the present invention may comprise a radical scavenger. As used herein the term radical scavenger refers to a species that can react with a radical, preferably a carbonate radical to convert the radical species by a series of fast reactions to a less reactive species. The radical scavenger is also preferably selected such that it is not an identical species as the alkalising agent and is present in an amount sufficient to reduce the damage to the hair during the coloring/bleaching process. The hair coloring compositions of the present invention may comprise from 0.1% to 10% by weight, preferably from 1% by weight to 7% by weight of a radical scavenger.

Suitable radical scavengers for use herein may be selected from the classes of alkanolamines, amino sugars, amino acids, esters of amino acids and mixtures thereof. Suitable compounds include 3-amino-1-propanol, 4-amino-1-butanol, 5-amino-1-pentanol, 1-amino-2-propanol, 1-amino-2-butanol, 1-amino-2-pentanol, 1-amino-3-pentanol, 1-amino-4-pentanol, 3-amino-2-methylpropan-1-ol, 1-amino-2-methylpropan-2-ol, 3-aminopropane-1,2-diol, glucosamine, N-acetylglucosamine, glycine, arginine, lysine, proline, glutamine, histidine, sarcosine, serine, glutamic acid, tryptophan, or mixtures thereof, or the salts, such as the potassium, sodium, or ammonium salts thereof, or mixtures thereof. The compositions may comprise glycine, sarcosine, lysine, serine, 2 methoxyethylamine, glucosamine, glutamic acid, morpholine, piperadine, ethylamine, 3 amino-1-propanol, or mixtures thereof.

### Solvent

The hair coloring compositions of the present invention may comprise a solvent. The solvent may be selected from water or a mixture of water and at least one organic solvent to dissolve the compounds that would not typically be sufficiently soluble in water. Suitable organic solvents for use herein include, but are not limited to: C1 to C4 lower alkanols (e.g., ethanol, propanol, isopropanol), aromatic alcohols (e.g. benzyl alcohol and phenoxyethanol); polyols and polyol ethers (e.g., carbitols, 2-butoxyethanol, propylene glycol, propylene glycol monomethyl ether, diethylene glycol monoethyl ether, monomethyl ether, hexylene glycol, glycerol, ethoxy glycol, butoxydiglycol, ethoxydiglycerol, dipropyleneglycol, polygylcerol), and propylene carbonate. When present, organic solvents are typically present in an amount ranging from 1% to 30%, by weight of the hair coloring compositions. The solvent may comprise water, ethanol, propanol, isopropanol, glycerol, 1,2-propylene glycol, hexylene glycol, ethoxy diglycol, or mixtures thereof.

### Thickeners

The hair coloring compositions may comprise a thickener in an amount sufficient to provide the composition with a viscosity so that it can be readily applied to the hair without unduly dripping off the hair and causing mess. Typically, such an amount will be at least 0.1%, in some embodiments, at least 0.5%, in other embodiments, at least 1%, by weight of the hair coloring composition. Preferred polymeric thickeners include a polymer thickener, comprising at least one polymer selected from associative polymers, polysaccharides, non-associative polycarboxylic polymers, and mixtures thereof.

As used herein, the expression “associative polymers” means amphiphilic polymers comprising both hydrophilic units and hydrophobic units, for example, at least one C8 to C30 fatty chain and at least one hydrophilic unit. Associative polymers are capable of reversibly combining with each other or with other molecules. Representative associative thickeners that may be used are associative polymers chosen from:

(i) nonionic amphiphilic polymers comprising at least one hydrophilic unit and at least one fatty-chain unit;
(ii) anionic amphiphilic polymers comprising at least one hydrophilic unit and at least one fatty-chain unit;
(iii) cationic amphiphilic polymers comprising at least one hydrophilic unit and at least one fatty-chain unit; and
(iv) amphoteric amphiphilic polymers comprising at least one hydrophilic unit and at least one fatty-chain unit.
The nonionic amphiphilic polymers comprising at least one fatty chain and at least one hydrophilic unit may, for example, be chosen from:
(1) celluloses modified with groups comprising at least one fatty chain; for example; hydroxyethylcelluloses modified with groups comprising at least one fatty chain chosen from alkyl, alkenyl and alkylaryl groups;
(2) hydroxypropyl guars modified with groups comprising at least one fatty chain;
(3) polyether urethanes comprising at least one fatty chain, such as C8-C30 alkyl or alkenyl groups;
(4) copolymers of vinylpyrrolidone and of fatty-chain hydrophobic monomers;
(5) copolymers of C1-C6 alkyl acrylates or methacrylates and of amphiphilic monomers comprising at least one fatty chain;
(6) copolymers of hydrophilic acrylates or methacrylates and of hydrophobic monomers comprising at least one fatty chain.

The anionic amphiphilic polymers comprising at least one hydrophilic unit and at least one fatty-chain unit, may, for example, be chosen from those comprising at least one fatty-chain allyl ether unit and at least one hydrophilic unit comprising an ethylenic unsaturated anionic monomeric unit, for example, a vinylcarboxylic acid unit and further, for example, chosen from units derived from acrylic acids, methacrylic acids and mixtures thereof, wherein the fatty-chain allyl ether unit corresponds to the monomer of formula below:

CH2=C(R1)CH2OBnR (I)

in which R1 is chosen from H and CH3, B is an ethyleneoxy radical, n is chosen from zero and integers ranging from 1 to 100, R is chosen from hydrocarbon-based radicals chosen from alkyl, alkenyl, arylalkyl, aryl, alkylaryl and cycloalkyl radicals, comprising from 8 to 30 carbon atoms, and, further, for example, from 10 to 24 carbon atoms and even further, for example, from 12 to 18 carbon atoms.
The anionic amphiphilic polymers may further be chosen, for example, from those comprising at least one hydrophilic unit of unsaturated olefinic carboxylic acid type, and at least one hydrophobic unit of the type such as a (C8-C30) alkyl ester or (C8-C30) oxyethylenated alkyl ester of an unsaturated carboxylic acid. The hydrophilic unit of unsaturated olefinic carboxylic acid type corresponds to, for example, the monomer of formula (II) below:

CH2=C(R1)COOH (II)

in which R1 is chosen from H, CH3, C2H5 and CH2COOH, i.e. acrylic acid, methacrylic, ethacrylic and itaconic acid units. And the hydrophobic unit of the type such as a (C8-C30) alkyl ester or (C8-C30) oxyethylenated alkyl ester of an unsaturated carboxylic acid corresponds to, for example, the monomer of formula (III) below:

CH2-C(R1)COOBnR2 (III)

in which R1 is chosen from H, CH3, C2H5 and CH2COOH (i.e. acrylate, methacrylate, ethacrylate and itaconate units), B is an ethyleneoxy radical, n is chosen from zero and integers ranging from 1 to 100, R2 is chosen from C8-C30 alkyl radicals, for example, C12-C22 alkyl radical.
Representative anionic amphiphilic polymers that can be used may further be cross-linked. The crosslinking agent can be a monomer comprising a group (IV)

CH2=C< (IV)

with at least one other polymerizable group whose unsaturated bonds are not conjugated with respect to one another. Mention may be made, for example, of polyallyl ethers such as polyallylsucrose and polyallyl pentaerythritol.

The cationic amphiphilic polymers used are, for example, chosen from quaternized cellulose derivatives and polyacrylates comprising amino side groups. The quaternized cellulose derivatives are, for example, chosen from quaternized celluloses modified with groups comprising at least one fatty chain, such as alkyl, arylalkyl and alkylaryl groups comprising at least 8 carbon atoms, and mixtures thereof, quaternized hydroxyethylcelluloses modified with groups comprising at least one fatty chain, such as alkyl, arylalkyl and alkylaryl groups comprising at least 8 carbon atoms, and mixtures thereof. The alkyl radicals borne by the above quaternized celluloses and hydroxyethylcelluloses, for example, contain from 8 to 30 carbon atoms. The aryl radicals, for example, are chosen from phenyl, benzyl, naphthyl and anthryl groups.

Among amphoteric amphiphilic polymers comprising at least one hydrophilic unit and at least one fatty-chain unit, mention may be made, for example, of methacrylamidopropyltrimethylammonium chloride/acrylic acid/C8-C30 alkyl methacrylate copolymers, wherein the alkyl radical is, for example, a stearyl radical.

Preferable associative polymeric thickeners for use herein comprise at least one hydrophilic unit which is unsaturated carboxylic acid or its derivative, and at least one hydrophobic unit which is a C8 to C30 alkyl ester or oxyethylenated C8-C30 alkyl ester of unsaturated carboxylic acid. The unsaturated carboxylic acid is preferably acrylic acid, methacrylic acid or itaconic acid. Examples can be made of materials sold under trade name Aculyn-22, Permulen TR1, Carbopol 2020, Carbopol Ultrez-21, Structure 2001, Structure 3001. Another preferable associative polymer for use in the polymer thickening systems of the present invention include polyether polyurethane, for example materials Aculyn-44, Aculyn-46. Another preferable associative polymer for use herein is cellulose modified with groups comprising at least one C8-C30 fatty chain, such as Natrosol Plus Grade 330 CS.

Non-associative cross-linked polycarboxylic polymers for use herein can be chosen, for example, from: (i) cross-linked acrylic acid homopolymers; (ii) copolymers of acrylic or (meth)acrylic acid and of C1-C6 alkyl acrylate or (meth)acrylate. Preferable polymers are Carbopol 980, 981, 954, 2984, 5984, Synthalen M/L/K, Aculyn-33.

The polysaccharides for use herein are, for example, chosen from glucans, modified and unmodified starches (such as those derived, for example, from cereals, for instance wheat, corn or rice, from vegetables, for instance yellow pea, and tubers, for instance potato or cassaya), amylose, amylopectin, glycogen, dextrans, celluloses and derivatives thereof (methylcelluloses, hydroxyalkylcelluloses, ethyl hydroxyethylcelluloses, and carboxymethylcelluloses), mannans, xylans, lignins, arabans, galactans, galacturonans, chitin, chitosans, glucuronoxylans, arabinoxylans, xyloglucans, glucomannans, pectic acids and pectins, alginic acid and alginates, arabinogalactans, carrageenans, agars, glycosaminoglucans, gum arabics, gum tragacanths, ghatti gums, karaya gums, carob gums, galactomannans, such as guar gums, and nonionic derivatives thereof (hydroxypropyl guar) and bio-polysaccharides, such as xanthan gums, gellan gums, welan gums, scleroglucans, succinoglycans and mixtures thereof.

For example, suitable polysaccharides are described in “Encyclopedia of Chemical Technology”, Kirk-Othmer, Third Edition, 1982, volume 3. pp. 896-900, and volume 15, pp. 439-458, in “Polymers in Nature” by E. A. MacGregor and C. T. Greenwood, published by John Wiley & Sons, Chapter 6, pp. 240-328,1980, and in “Industrial Gums—Polysaccharides and their Derivatives”, edited by Roy L. Whistler, Second Edition, published by Academic Press Inc., the content of these three publications being entirely incorporated by reference.

The polysaccharide is preferably a bio-polysaccharide, particularly preferable are bio-polysaccharides selected from xanthan gum, gellan gum, welan gum, scleroglucan or succinoglycan, for example materials such as Keltrol T, Rheozan. Another preferable polysaccharide is hydroxypropyl starch derivative, particularly preferable hydroxypropyl starch phosphate, for example Structure XL®.

Suitable for use herein are salt tolerant thickeners, including but not limited to: xanthan, guar, hydroxypropyl guar, scleroglucan, methyl cellulose, ethyl cellulose (as AQUACOTE), hydroxyethyl cellulose (NATROSOL), carboxymethyl cellulose, hydroxypropylmethyl cellulose, microcrystalline cellulose, hydroxybutylmethyl cellulose, hydroxypropyl cellulose (as KLUCEL), hydroxyethyl ethyl cellulose, cetyl hydroxyethyl cellulose (as NATROSOL Plus 330), N-vinylpyrollidone (as POVIDONE), Acrylates/Ceteth-20 Itaconate Copolymer (as STRUCTURE 3001), hydroxypropyl starch phosphate (as STRUCTURE ZEA), polyethoxylated urethanes or polycarbamyl polyglycol ester (e.g. PEG-150/Decyl/SMDI copolymer (as ACULYN 44), PEG-15(0Stearyl/SMDI copolymer (as ACULYN 46), trihydroxystearin (available as THIXCIN), acrylates copolymer (e.g. as ACULYN 33) or hydrophobically modified acrylate copolymers (e.g. Acrylates/Steareth-20 Methacrylate Copolymer (as ACULYN 22), acrylates/steareth-20 methacrylate crosspolymer (as ACULYN 88), acrylates/vinyl neodecanoate crosspolymer (available as ACULYN 38), acrylates/beheneth-25 methacrylate copolymer (as ACULYN 28), acrylates/C10-30 alkyl acrylate crosspolymer (as Carbopol® ETD 2020), non-ionic amphophilic polymers comprising at least one fatty chain and at least one hydrophilic unit selected from polyether urethanes comprising at least one fatty chain, and blends of Ceteth-10 phosphate, Di-cetyl phosphate and Cetearyl alcohol (as CRODAFOS CES).

Preferred thickeners for use in the first developer component include acrylates copolymer, hydrophobically modified acrylate copolymers (e.g. Acrylates/Steareth-20 Methacrylate Copolymer) and mixtures thereof. Preferred thickeners polymers for use in the dye component include, blends of Ceteth-10 phosphate, Di-cetyl phosphate and Cetearyl alcohol (as CRODAFOS CES).

### Gel Network Thickener System

The hair coloring compositions of the present invention may comprise at at least one gel network thickener system. Said system may comprise at least one low HLB surfactant and/or amphophile having a high melting point, and at least one additional second surfactant as specified hereinafter. Suitable gel network thickener systems are disclosed in PCT application WO2006/060598A1, the contents of which are hereby incorporated by reference.

Said low HLB surfactant and/or amphophile may have preferably an HLB of 6 or less and melting point of at least 30° C. It may be selected from the group consisting of cetyl, stearyl, cetostearyl or behenyl alcohols, steareth-2, glycerol monostearate and mixtures thereof. Said second surfactant may be anionic, non-ionic or cationic. Anionic surfactants may be selected from the group consisting of alkyl ether phosphates having in average 1-3 ethylene oxide units and comprising an alkyl radical comprising from 8 to 30 carbon atoms. Said non-ionic surfactants may be selected from the group consisting of those comprising one or more polyethyleneoxide chain including polyoxyethylene alkyl ethers having from 100 to 200 ethylene oxide units (e.g. steareth-100, steareth-150). Said cationic surfactant may be selected from the group consisting of behentrimonium chloride, behenamidopropyltrimonium methosulfate, stearamidopropyltrimonium chloride, arachidtrimonium chloride and mixtures thereof. A preferred gel network thickening system comprises fatty alcohols having 14 to 30 carbon atoms (cetyl and/or stearyl alcohol) and alkyl ether phosphates (e.g. from 1 to 3 ethylene oxide units). The compositions of the present invention may comprise a total amount of gel network thickening system of from 2% to 10% by weight of the hair coloring composition. The weight ratio of the low HLB surfactants to the second specified surfactants is preferably from 10:1 to 1:1.

### Carbonate Ion Source

The hair coloring compositions of the present invention may comprise a source of carbonate ions, carbamate ions, or hydrogen carbonate ions, in a sufficient amount to reduce damage to the hair during the coloring process. Such an amount may range from 0.1% to 15%, or from 0.1% to 10%, or from 1% to 7%, by weight of the hair coloring composition. Suitable sources for the ions include but are not limited to: sodium carbonate, sodium hydrogen carbonate, potassium carbonate, potassium hydrogen carbonate, guanidine carbonate, guanidine hydrogen carbonate, lithium carbonate, calcium carbonate, magnesium carbonate, barium carbonate, ammonium carbonate, ammonium hydrogen carbonate and mixtures thereof. In some embodiments, the source of carbonate ions is sodium hydrogen carbonate, potassium hydrogen carbonate, or mixtures thereof. The source of carbonate ions may be ammonium carbonate, ammonium hydrogen carbonate, or mixtures thereof.

### Conditioning Agent

The hair coloring compositions may comprise a conditioning agent. The conditioning agent may be present in any component or in any combination thereof. Conditioning agents suitable for use herein are selected from silicone materials, amino silicones, C6 to C30 fatty alcohols, polymeric resins, polyol carboxylic acid esters, cationic polymers, cationic surfactants, insoluble oils and oil derived materials and mixtures thereof. Additional materials include mineral oils and other oils such as glycerin and sorbitol. Particularly useful conditioning materials are cationic polymers. Conditioners of cationic polymer type can be chosen from those comprising units of at least one amine group chosen from primary, secondary, tertiary and quaternary amine groups that may either form part of the main polymer chain, or be borne by a side substituent that is directly attached to the main polymer chain, described hereinafter. The conditioning agent will generally be used at levels of from 0.05% to 20%, preferably of from 0.1% to 15%, more preferably of from 0.2% to 10%, even more preferably of from 0.2% to 2% by weight of the hair coloring composition. The conditioning agent may be included in a separate pre- or post-treatment composition.

Silicones can be selected from polyalkylsiloxane oils, linear polydimethylsiloxane oils containing trimethylsilyl or hydroxydimethylsiloxane endgroups, polymethylphenylsiloxane, polydinethylphenylsiloxane or polydimethyldiphenylsiloxane oils, silicone resins, organofunctional siloxanes having in their general structure one or a number of organofunctional group(s), the same or different, attached directly to the siloxane chain or mixtures thereof. Said organofunctional group(s) are selected from: polyethyleneoxy and/or polypropyleneoxy groups, (per)fluorinated groups, thiol groups, substituted or unsubstituted amino groups, carboxylate groups, hydroxylated groups, alkoxylated groups, quaternium ammonium groups, amphoteric and betaine groups. The silicone can either be used as a neat fluid or in the form of a pre-formed emulsion.

Suitable silicones include silicones containing groups that may be ionized into cationic groups, for example aminosilicones containing at least 10 repeating siloxane (Si(CH₃)₂—O) units within the polymer chain, with either terminal, graft, or a mixture of terminal and graft aminofunctional groups. Example functional groups are not limited to aminoethylaminopropyl, aminoethylaminoisobutly, aminopropyl. In the case of graft polymers, the terminal siloxane units can be (CH₃)₃Si—O, R₁₂(CH₃)₂Si—O, where R₁₂ can be either OH or OR₁₃, where R₁₃ is a C1-C8 alkyl group, or a mixture of both terminal groups. These silicones are also available as preformed emulsions. Commercially available aminosilicones include DC-2-8566, DC 7224, DC-2-8220, SF1708. SM2125, Wacker Belsil ADM 653/1100/1600/652/6057E/8020, DC929, DC939, DC949, SM2059. Aminosilicones may also contain additional functional groups. Additional functional groups can include polyoxyalkylene, the reaction product of amines and carbinols, and alky chains. For example products know as methoxy PEG/PPG-7/3 Aminopropyl Dimethicone, such as Abil Soft AF100, or products know as Bis (C13-15 Alkoxy) PG Amodimethicone, such as DC 8500.

Cationic polymers suitable for use herein can be chosen from those comprising units of at least one amine group chosen from primary, secondary, tertiary and quaternary amine groups that may either form part of the main polymer chain or be borne by a side substituent that is directly attached to the main polymer chain. Such cationic polymers generally have a number average molecular mass ranging from 500 to about 5×10⁶, or more preferably from 1000 to 3×10⁶. Preferably the cationic polymers are selected from polymers of the polyamine, polyamino amide and polyquaternary ammonium type.

Polymers of the polyamine, polyamino amide and polyquaternary ammonium type that may be used include but are not limited to:

1) Homopolymers and copolymers derived from acrylic or methacrylic esters or amides. Copolymers of these polymers can also comprise at least one unit derived from comonomers which may be chosen from the family of acrylamides, methacrylamides, diacetone acylamides, acrylamides and methacrylicamides substituted on the nitrogen with at least one group chosen from lower (C1-C4) alkyls, acrylic and methacrylic acids and esters thereof, vinlylactams such as vinlypyrrolidone and vinylcaprolactam, and vinyl esters.
Copolymers of acrylamide and of methacryloyloxyethyltrimethylammonium methosulfate, examples of which include polymers known as Polyquaternium-5 (herein referred as PQ”), such as Reten 210/220/230/240/1104/1105/1006, Merquat 5/5SF.

Copolymers of vinylpyrrolidone and dimethylaminopropyl methacrylamide, examples of which include polymers known as PQ-28, such as Gafquat HS-100. Copolymers of vinyl pyrrolidone and dialkyaminoalkyl acrylates or methacrylates, examples of which include polymers known as PQ-11, such as Gafquat 440/734/755/755N, Luviquat PQ11 PM, Polyquat-11 SL. Copolymers vinylpyrrolidone, dimethylaminopropyl methacrylamide and methacryloylaminopropyl lauryldimonium chloride, examples of which include polymers known as PQ-55, such as Styleze W-20. Copolymers of acrylic acid, acrylamide and methacrylamidopropyltrimonium chloride, examples of which include polymers known as PQ-53, such as Merquat 2003. Copolymers of dimethyaminopropylacrylate (DMAPA), acrylic acid and acrylonitrogens and diethyl sulfate, examples of which include polymers known as PQ-31, such as Hypan QT100. Copolymers of acrylamide, acrylamidopropyltrimonium chloride, 2-aminopropylacrylamide sulfonate, and dimethyaminopropylacrylate (DMAPA), examples of which include polymers known as PG-43, such as Bozequat 4000. Copolymers of acrylic acid, methylacrylate and methacrylamidopropyltrimonium chloride, examples of which include polymers known as PQ-47, such as Merquat 2001/2001N. Copolymers of methacryloyl ethyl betaine, 2-hydroxyethyl methacrylate and methacryloyl ethyl trimethyl ammonium chloride, examples of which include polymers known as PQ-48, such as Plascize L-450. Copolymers of acrylic acid diallyl dimethyl ammonium chloride and acrylamide, examples of which include polymers known as PQ-39, such as Merquat 3330/3331. Further examples include copolymers of methacrylamide methacrylamido-propyltrimonium and methacryloylethyltrimethyl ammonium chloride and their derivatives, either homo or copolymerised with other monomers, examples of which include polymers known as: PQ-8, PQ-9, PQ-12, PQ-13; PQ-14, PQ-15, such as Rohagit KF720 F, PQ-30, such as Mexomere PX, PQ-33, PQ-35, PQ-36, such as Plex 3074 L, PQ-45, such as Plex 3073L. PQ-49, such as Plascize L-440, PQ50 such as Plascize L-441, PQ-52.

2) Cationic polysaccharides, such as cationic celluloses and cationic galactomannan gums. Among the cationic polysaccharides that maybe mentioned, for example, are cellulose ether derivatives comprising quaternary ammonium groups and cationic cellulose copolymers or cellulose derivatives grafted with a water-soluble quaternary ammonium monomer and cationic galactomannan gums. Examples include but are not limited to: Copolymers of hydroxyethylcelluloses and diallyldimethyl ammonium chlorides, examples of which include polymers known as PQ-4, such as Celquat L200/H100. Copolymers of hydroxyethylcelluloses and a trimethyl ammonium substituted epoxide, examples of which include polymers known PQ-10, such as AEC Polyquaternium-10, Catinal C-100/HC-35/HC-100/HC-200/LC-100/LC-200, Celquat SC-240C/SC-230M, Dekaquat 400/3000, Leogard GP, RITA Polyquta 400/3000, UCARE Polymer JR-125/JR-400/JR-30M/LK/LR400/LR30M. Copolymers of hydroxyethylcelluloses and lauryl dimethyl ammonium substituted epoxides, examples of which include polymers known as PQ-24, such as Quatrisoft polymer LM-200. Derivatives of Hydroxypropyl Guar, examples of which include polymers known as Guar Hydroxypropyltrimonium Chloride, such as Catinal CG-100/CG-200, Cosmedia Guar C-261N/C-261N/C-261N, DiaGum P 5070, N-Hance Cationic Guar, Hi-Care 1000, Jaguar C-17/C-2000/C-13S/C-14S/Excel, Kiprogum CW, Kiprogum NGK. Hydroxypropyl derivatives of Guar Hydroxypropyltrimonium Chloride, examples of which include polymers known as Hydroxypropyl Guar Hydroxypropyltrimonium Chloride, such as Jaguar C-162.
3) Polyamino amide derivatives resulting from the condensation of polyalkylene polyamines with polycarboxylic acids followed by alkylation with difunctional agents. Among the derivative, mention may be made for example to adipic acid/dimethylaminohydroxypropyl/diethylenetriamine.
4) Polymers obtained by reaction of a polyalkylene polyamine comprising two primary amines groups and at last one secondary amine group with a decarboxylic acid chosen from diglycolic acids and saturated aliphatic dicarboxylic acids comprising from 3 to 8 carbon atoms. Non-limiting examples of such derivatives include the adipic acid/epxoypropyl/diethylenetriamine.
5) Cyclopolymers of dialkdiallylamine or of dialkyldiallyammonium, among which polymers mention may be made of: Dimethyldiallyammonium chloride polymers, examples of which include polymers known as PQ-6, such as Merquat 100, Mirapol 100, Rheocare CC6, AEC polyquaternium-6, Agequat 400, Conditioner P6, Flocare C106, Genamin PDAC, Mackernium 006. Copolymers of acrylamides and dimethyldiallylammonium chlorides monomers, examples of which include polymers known as PQ-7, such as AEC Polyquaternium-7, Agequat-5008/C-505, Conditioner P7, Flocare C107, Mackernium 007/007S, ME Polymer 09W, Merquat 550/2200/S, Mirapol 550, Rheocare CC7/CCP7, Salcare HSP-7/SC10/Super 7. Copolymers of dimethyldiallylammoniumchlorides and acrylic acids, examples of which include polymers known as polyquaternary-22, such as Merqual 280/Merquat 295.
6) Quaternary diammonium polymers comprising repeat units corresponding to [—N+(R1)(R2)-A1-N+(R3)(R4)-B1-][2X—], in which R1, R2, R3 and R4, which may be identical or different, are chosen from aliphatic, alicyclic and arylaliphatic radicals comprising from 1 to 20 carbon atoms and from lower hydroxyalkylaliphatic radicals, or R1, R2, R3 and R4, together or separately, constitute, with the nitrogen atoms to which they are attached, heterocycles optionally comprising a second heteroatom other then nitrogen, or R1, R2, R3 and R4, are chosen from liner or branched C1-C6 alkyl radicals substituted with at least one group chosen from nitrile, ester, acyl and amide groups and groups of —CO—O-R5-D and —CO—NH—R5-D wherein R5 is chosen from alkylene groups and D is chosen from quaternary ammonium groups. A1 and B1, which may be identical or different, are chosen from linear and branched, saturated or unsaturated polymethylene groups comprising 2 to 20 carbon atoms. The polymethylene groups may comprise, linked to or intercalated in the main ring, at least one entity chosen from aromatic rings, oxygen and sulphur atoms and sulphoxide, sulphone, disulphide, amino, alkylamino, hydroxyl, quaternary, ammonium, ureido, amide and ester groups, and X— is an anion derived from inorganic and organic acids. D is chosen from a glycol residue, a bis-secondary diamine residue, a bis-primary diamine residue or a ureylene group. An examples of which include polymers known as Hexadimethrine chloride, where R1, R2, R3 and R4 are each methyl radicals, A1 is (CH2)3 and B1 is (CH2)6 and X═Cl. Further examples of which include polymers known as PQ-34 where R1 and R2 are ethyl radicals and R3 and R4 are methyl radicals and A1 is (CH2)3 and B1 is (CH2)3 and X═Br, such as Mexomere PAX.
7) Polyquaternary ammonium polymers comprising repeating units of formula [—N+(R6)(R7)-(CH2)r-NH—CO—(CH2)q-(CO)t-NH—(CH2)s-N+(R8)(R9)-A-][2X—.] in which R6, R7, R8 and R9 which may be identical or different, are chosen from a hydrogen atom and a methyl, ethyl, propyl, hydroxyethyl, hydroxypropyl, and —CH2CH2(OCH2CH2)pOH radicals, wherein p is equal to 0 or an integer ranging from 1 to 6, wherein R6, R7, R8 and R9 do not all simultaneously represent a hydrogen atom. R and s which maybe identical or different are each an integer ranging from 1 to 6, q is equal to 0 or an integer ranging from 1 to 34 and X— is anion such as a halide. T is an integer chosen to be equal to 0 or 1. A is chosen from divalent radicals such as —CH2-CH2-O—CH2-CH2-. Examples of which include: Polymers known as PQ-2, where r=s=3, q=0, t=0, R6, R7, R8 and R9 are methyl groups, and A is —CH2-CH2-O-CH2-CH2, such as Ethpol PQ-2, Mirapol A-15. Polymers known as PQ-17 where r=s=3, q=4, t=1 R6, R7, R8 and R9 are methyl groups, and A is —CH2-CH2-O-CH2-CH2. Polymers known as PQ-18, where r=s=3, q=7, t=1 R6, R7, R8 and R9 are methyl groups, and A is —CH2-CH2-O—CH2-CH2. Polymers known as the block copolymer formed by the reaction of Polyquaternium-2 with Polyquaternium-17, known as PQ-27, such as Mirapol 175.
8) Copolymers of vinylpyrrolidones and of vinylimidazoles and optionally vinylcaprolactums, examples of which include polymers known as PQ-16 formed from methylvinylimidazolium chlorides and vinylpyrrolidones, such as Luviquat FC370/FC550/FC905/HM-552. Or copolymers of vinylcaprolactams and vinylpyrrolidones with methylvinylimidazolium methosulfates, examples of which include polymers known as PQ-46, such as Luviquat Hold. Or copolymers of vinylpyrrolidones and quaternized imidazolines, examples of which include polymers known PQ-44, such as Luviquat Care.
9) Polyamines such as the product Polyquart H sold by Cognis under the reference name polyethylene glycol (15) tallow polyamine in the CTFA dictionary.
10) Cross linked methacryloyloxy(C1-C4)alkyltri(C1-C4)alkylammonium salt polymers such as the polymers obtained by homopolymerisation of dimethylaminoethyl methacrylates quaternized with methyl chloride, or by copolymerisation of acrylamides with dimethylaminoethyl methacrylates quaternized with methyl chloride, the homo or copolymerisation being followed by crosslinking with a compound comprising olefinic unsaturation, such as methylenebisacrylamides, examples of which include polymers known as PQ-37, such as Synthalen CN/CR/CU, or as a dispersion in another media such as Salcare SC95/SC96, Rheocare CTH(E). Or in another example of which include polymers known as PQ-32, or when sold as a dispersion in mineral oil such as Salcare SC92.
11) Further examples of cationic polymers include polymers known as PQ-51, such as Lipidure-PMB, as PQ-54, such as Qualty-Hy, as PQ-56 such as Hairrol UC-4, and as PQ-87 such as Luviquat sensation.
12) Silicone polymers comprising cationic groups and/or groups which may be ionised into cationic groups. For example: cationic silicones of the general formula (R10-N+(CH3)2)-R11-(Si(CH3)2-O)x-R11-(N+(CH3)2)-R10), where R10 is an alkyl derived from coconut oil, and R11 is (CH2CHOCH2O(CH2)3 and x is a number between 20 and 2000, examples of which include polymers known as Quaternium 80, such as Abil Quat 3272/3474.
Silicones containing groups which may be ionised into cationic groups, for example aminosilicones containing at least 10 repeating siloxane —(Si(CH3)2-O) units within the polymer chain, with either terminal, graft or a mixture of terminal and graft aminofunctional groups. Example functional groups are not limited to aminoethylaminopropyl, aminoethylaminoisobutly, aminopropyl. In the case of graft polymers, the terminal siloxane units can either be (CH3)3Si—O or R12(CH3)2Si—O, where R12 can be either OH or OR13, where R13 is a C1-C8 alky group, or a mixture of both functional terminal groups. These silicones are also available as preformed emulsions. Polymer with terminal siloxane units of (CH3)3Si—O examples of which include polymers known as trimethylsilylamodimethicone, such as DC-2-8566, DC 7224, DC-2-8220, SF1708, SM 2125, Wacker Belsil ADM 653. Further examples include polymers with terminal siloxane units of (R12O)(CH3)2Si—O where R12 can be either OH or OR13, where R13 is a C1-C8 alky group, or a mixture of both functional terminal groups, known as amodimethicone, such as Wacker Belsil ADM 1100/ADM 1600/ADM 652/ADM 6057E/ADM 8020, DC929, DC939, DC949, SM2059.
Silicones containing groups which may be ionised into cationic groups—for example silicones containing at least 10 repeating siloxane —(Si(CH3)2-O) units within the polymer chain, with either terminal, graft or a mixture of terminal and graft aminofunctional groups, together with additional functional groups. Additional functional groups can include polyoxyalkylene, the reaction product of amines and carbinols, alky chains. For example products known as methoxy PEG/PPG-7/3 Aminopropyl Dimethicone, such as Abil Soft AF100. For example products know Bis (C13-15 Alkoxy) PG Amodimethicone, such as DC 8500.

The hair coloring compositions and/or components of the present invention may comprise at least 0.2%, or from 0.5% to 2% by weight of the composition of a cationic polymer.

### Surfactants

The hair coloring compositions according to the present invention may comprise one or more surfactants. Surfactants suitable for use herein generally have a lipophilic chain length of from 8 to 30 carbon atoms and can be selected from anionic, nonionic, amphoteric and cationic surfactants and mixtures thereof. The total level of surfactant may be from 2% to 30%, or from 8% to 25%, or from 10% to 20% by weight of the hair coloring composition.

The developer component may comprise from 0 to 5% by weight of surfactant. The dye component may comprise from 0 to 10% by weight of surfactant.

Anionic surfactants, where may be present in the range of from 0.1% to 20%, or from 0.1% to 15%, or from 5% to 15% by weight of the hair coloring composition; amphoteric or nonionic surfactants, may independently be present is in the range of from 0.1% to 15%, or from 0.5% to 10%, or from 1% to 8% by weight of the hair coloring composition.

Suitable anionic surfactants, which can be used, alone or as mixtures, include salts (such as alkaline salts, for example, sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates: alkyl sulphonates, alkyl phosphates, alkylamide sulphonates, alkylaryl sulphonates, a-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates and N-acyltaurates. The alkyl or acyl radical of all of these various compounds, for example, comprises from 8 to 24 carbon atoms, and the aryl radical, for example, is chosen from phenyl and benzyl groups. Among the anionic surfactants, which can also be used, mention may also be made of fatty acid salts such as the salts of oleic, ricinoleic, palmitic and stearic acids, coconut oil acid or hydrogenated coconut oil acid; acyl lactylates in which the acyl radical comprises from 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof. Anionic derivatives of polysaccharides, for example carboxyalkyl ether of alkyl polyglucosides, can be also used.

The nonionic surfactants are compounds that are well known (see, for example, in this respect “Handbook of Surfactants” by M. R. Porter, published by Blackie & Son (Glasgow and London), 1991, pp. 116-178). They can be chosen, for example, from polyethoxylated, polypropoxylated and polyglycerolated fatty acids, alkyl phenols, a-diols and alcohols comprising a fatty chain comprising, for example, from 8 to 18 carbon atoms, it being possible for the number of ethylene oxide or propylene oxide groups to range, for example, from 2 to 200 and for the number of glycerol groups to range, for example, from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide and their momothanolamine and diethanolamine derivatives, polyglycerolated fatty amides, for example, comprising on average from 1 to 5, and such as from 1.5 to 4, glycerol groups; polyethoxylated fatty amines such as those containing from 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

The amphoteric surfactants can be chosen, for example, from aliphatic secondary and tertiary amine derivatives in which the aliphatic radical is chosen from linear and branched chains comprising from 8 to 22 carbon atoms and comprising at least one water-soluble anionic group (for example carboxylate, sulphonate, sulphate, phosphate or phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines. Among the amine derivatives, mention may be made of the products sold under the name Miranol, as described, for example, in U.S. Pat. Nos. 2,528,378 and 2,781,354 and having the structures of: R₂—CONHCH₂CH₂—N⁺(R₃)(R₄)(CH₂COO⁻), (VT) in which: R₂ is chosen from alkyl radicals derived from an acid R₂—COOH present in hydrolysed coconut oil, and heptyl, nonyl and undecyl radicals, R₃ is a 5-hydroxyethyl group and R₄ is a carboxymethyl group; and of R₅—CONHCH₂CH₂—N(B)(C) (VII) wherein B represents —CH₂CH₂OX′, C represents —(CH₂)_{z}—Y′, with z=1 or 2, X′ is chosen from the —CH₂CH₂—COOH group and a hydrogen atom, Y′ is chosen from —COOH and —CH₂—CHOH—SO₃H radicals, R₅ is chosen from alkyl radicals of an acid R₅—COOH present in coconut oil or in hydrolysed linseed oil, alkyl radicals, such as C₇, C₉, C₁₁ and C₁₃ alkyl radicals, a C₁₇ alkyl radical and its iso form, and unsaturated C₁₇ radical. These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid. Salts of diethyl aminopropyl cocoaspartamid can be also used.

The cationic surfactants may be chosen from: A) the quaternary ammonium salts of general formula (VIII) below:

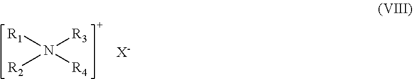

wherein X⁻ is an anion chosen from halides (chloride, bromide and iodide), (C₂-C₆)alkyl sulphates, such as methyl sulphate, phosphates, alkyl and alkylaryl sulphonates, and anions derived from organic acids, such as acetate and lactate, and
i) the radicals R₁ to R₃, which may be identical or different, are chosen from linear and branched aliphatic radicals comprising from 1 to 4 carbon atoms, and aromatic radicals such as aryl and alkylaryl. The aliphatic radicals can comprise at least one hetero atom such as oxygen, nitrogen, sulphur and halogens. The aliphatic radicals are chosen, for example, from alkyl, alkoxy and alkylamide radicals, R₄ is chosen from linear and branched alkyl radicals comprising from 16 to 30 carbon atoms. The cationic surfactant is, for example, a behenyltrimethylammonium salt (for example chloride).
ii) the radicals R₁ and R₂, which may be identical or different, are chosen from linear and branched aliphatic radicals comprising from 1 to 4 carbon atoms, and aromatic radicals such as aryl and alkylaryl. The aliphatic radicals can comprise at least one hetero atom such as oxygen, nitrogen, sulphur and halogens. The aliphatic radicals are chosen, for example, from alkyl, alkoxy, alkylamide and hydroxyalkyl radicals comprising from 1 to 4 carbon atoms; R₃ and R₄, which may be identical or different, are chosen from linear and branched alkyl radicals comprising from 12 to 30 carbon atoms, the said alkyl radicals comprise at least one function chosen from ester and amide functions. R₃ and R₄ are chosen, for example, from (C₁₂-C₂₂)alkylamido(C₂-C₆)alkyl and (C₁₂-C₂₂) alkylacetate radicals. The cationic surfactant is, for example, a dicetyldimethyl ammonium salt (for example chloride);
B)—the quaternary ammonium salts of imidazolinium, such as that of formula (IX) below:

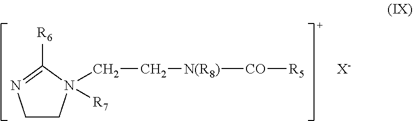

in which R₅ is chosen from alkenyl and alkyl radicals comprising from 8 to 30 carbon atoms, for example fatty acid derivatives of tallow,
R₆ is chosen from a hydrogen atom. C₁-C₄ alkyl radicals and alkenyl and alkyl radicals comprising from 8 to 30 carbon atoms, R₇ is chosen from C₁-C₄ alkyl radicals, R₈ is chosen from a hydrogen atom and C₁-C₄ alkyl radicals, and X⁻ is an anion chosen from halides, phosphates, acetates, lactates, alkyl sulphates, alkyl sulphonates and alkylaryl sulphonates.
R₅ and R₆ may be, for example, a mixture of radicals chosen from alkenyl and alkyl radicals comprising from 12 to 21 carbon atoms, such as fatty acid derivatives of tallow, R₇ is methyl and R₈ is hydrogen. Such a product is, for example, Quaternium-27 (CTFA 1997) or Quaternium-83 (CTFA 1997), which are sold under the names “Rewoquat®” W75, W90, W75PG and W75HPG by the company Witco,
C)—the diquaternary ammonium salts of formula (X):

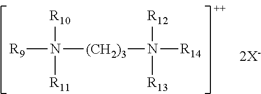

in which R₉ is chosen from aliphatic radicals comprising from 16 to 30 carbon atoms, R₁₀, R₁₁, R₁₂, R₁₃ and R₁₄, which may be identical or different, are chosen from hydrogen and alkyl radicals comprising from 1 to 4 carbon atoms, and X⁻ is an anion chosen from halides, acetates, phosphates, nitrates and methyl sulphates. Such diquaternary ammonium salts, for example, include propanetallowdiammonium dichloride; and
D)—the quaternary ammonium salts comprising at least one ester function, of formula (XI) below:

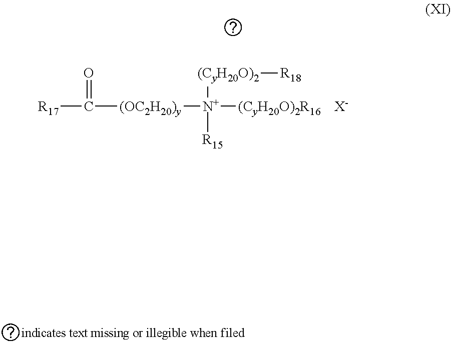

in which:
R15 is chosen from C₁-C₆ alkyl radicals and C1-C6 hydroxyalkyl and dihydroxyalkyl radicals; R16 is chosen from: a radical R19C(O)—, linear and branched, saturated and unsaturated C1-C22 hydrocarbon-based radicals R20, and a hydrogen atom, R18 is chosen from: a radical R21C(O)—, linear and branched, saturated and unsaturated C1-C6 hydrocarbon-based radicals R22, and a hydrogen atom. R17, R19 and R21, which may be identical or different, are chosen from linear and branched, saturated and unsaturated C7-C21 hydrocarbon-based radicals; n, p and r, which may be identical or different, are chosen from integers ranging from 2 to 6; y is chosen from integers ranging from 1 to 10: x and z, which may be identical or different, are chosen from integers ranging from 0 to 10; X— is an anion chosen from simple and complex, organic and inorganic anions; with the proviso that the sum x+y+z is from 1 to 15, that when x is 0, then R16 is R20 and that when z is 0, then R18 is R22.

The ammonium salts of formula (XI) can be used, in which: R15 is chosen from methyl and ethyl radicals, x and y are equal to 1; z is equal to 0 or 1; n, p and r are equal to 2; R16 is chosen from: a radical R19C(O)—, methyl, ethyl and C14-C22 hydrocarbon-based radicals, and a hydrogen atom; R17, R19 and R21, which may be identical or different, are chosen from linear and branched, saturated and unsaturated C7-C21, hydrocarbon-based radicals; R18 is chosen from: a radical R21C(O)— and a hydrogen atom. Such compounds are sold, for example, under the names Dehyquart by the company Cognis, Stepanquat by the company Stepan, Noxamium by the company Ceca, and Rewoqual WE 18 by the company Rewo-Witco.

### Viscosity

The developer component and the dye component may be, independently from one another, prepared as so called thin liquids or creams.

Each of the hair coloring composition A B and C may have a viscosity which induces a shear stress of from 20 to 200 Pa at 10 s⁻¹ as measured according to the viscosity test method.

### Application Means

The hair coloring compositions A, B and C may be applied to the hair with a brush and bowl applicator or with the hands and fingers of the user, or with a container to which a nozzle or a separate applicator device such as a comb is attached.

The application means may also include means which assist in achieving particular effects such as highlighting such as highlighting combs, brushes and tools, foils and highlighting caps. Additional application means technology can be used to assist in the penetration of the product into the hair. Examples of such technology include heating devices, ultraviolet light devices and ultrasound devices.

### Viscosity Test Method:

The viscosity of a composition is measured using a TA Instruments AR 20(00 Rheometer or equivalent device equipped with a Peltier plate and a 6 cm flat acrylic plate with cross hatchings. The instrument is calibrated according to the manufacturer's instructions and the Peltier plate is set at 25.0° C. The cone is raised to a position approximately 4.5 cm above the plate.

Immediately after the mixing, approximately 10 g of the mixture is transferred gently onto the center of the Peltier plate using a spatula. The cone is lowered to obtain the specified gap between the tip of the cone and the upper surface of the Peltier plate. The gap setting is specified by the manufacturer of the cone and is typically approximately 1000 microns. The rheometer is programmed to operate in rotational mode with the shear stress ramped from 0.1 to 600 Pa over a period of 4 minutes, termination at 1000 reciprocal seconds. Rotation is initiated immediately after the specified gap is established. Viscosity data collected during the measurement period are shear stress (Pa) plotted as a function of shear rate (s⁻¹).

### Experimental Data

1) Consumer Test Data

19 home hair color kit users who were frustrated by their grey root line received one of two versions (Medium brown or medium Blonde) of kits according to the invention depending on their starting hair type. After using the kit of the invention they were asked to compile a questionnaire 5-6 weeks after coloring and were asked to compare the results to the typical results they obtained previously at home with conventional products and to say if it was worse, equal or better in terms of disguising their grey root line. The average results of the questions asked was as follows:

### Worse: 0%

### Equal: 17%

### Better: 83%

The formulations tested in the kit were as follows:

### Developer Component

| | | Ingredient |
|---|---|---|
| | | Concentration |
| | CAS | in Finished |
| Ingredient INCI Name | Number | Product wt % |
| WATER Up to 100% | | |
| ACRYLATES COPOLYMER | 25212-88-8 | 2.520 |
| HYDROGEN PEROXIDE | 7722-84-1 | 1.900 |
| ETIDRONIC ACID | 2809-21-4 | 0.048 |
| DISODIUM EDTA | 139-33-3 | 0.036 |
| ACRYLATES/STEARETH- | 70879-60-6 | 0.030 |
| 20 METHACRYLATE | | |
| COPOLYMER | | |
| SIMETHICONE | 67762-90-7 | 0.005 |

### Dye Component—Medium Blonde

| | | Ingredient |
|---|---|---|
| | | Concentration |
| | CAS | in Finished |
| Ingredient INCI Name | Number | Product wt % |
| PROPYLENE GLYCOL | 57-55-6 | 7.63 |
| AMMONIUM HYDROXIDE | 1336-21-6 | 4.30 |
| CETEARYL ALCOHOL | 67762-27-0 | 2.10 |
| TRISODIUM ETHYLENEDIAMINE | 178949-82-1 | 1.29 |
| DISUCCINATE | | |
| AMMONIUM SULFATE | 7783-20-2 | 1.00 |
| SODIUM SULFITE | 7757-83-7 | 0.40 |
| DICETYL PHOSPHATE | 2197-63-9 | 0.37 |
| CETETH-10 PROSPHATE | 50643-20-4 | 0.29 |
| STEARETH-200 | 9005-00-9 | 0.25 |
| ASCORBIC ACID | 50-81-7 | 0.30 |
| SODIUM HYDROXIDE | 1310-73-2 | 0.13 |
| DISODIUM EDTA | 139-33-3 | 0.09 |
| XANTHAN GUM | 11138-66-2 | 0.04 |
| 2-METHOXYMETHYL-P- | 337906-36-2 | 0.310 |
| PHENYLENEDIAMINE | | |
| RESORCINOL | 108-46-3 | 0.158 |
| 2-METHYLRESORCINOL | 608-25-3 | 0.090 |
| N,N-BIS(2-HYDROXYETHYL)-P- | 54381-16-7 | 0.043 |
| PHENYLENEDIAMINE SULFATE | | |
| M-AMINOPHENOL | 591-27-5 | 0.018 |
| PERFUME | | 0.800 |
| WATER Up to 100% | | |

### Dye Component—Medium Brown

| | | Ingredient |
|---|---|---|
| | | Concentration |
| | CAS | in Finished |
| Ingredient INCI Name | Number | Product wt % |
| PROPYLENE GLYCOL | 57-55-6 | 7.63 |
| AMMONIUM HYDROXIDE | 1336-21-6 | 3.98 |
| CETEARYL ALCOHOL | 67762-27-0 | 2.10 |
| TRISODIUM ETHYLENEDIAMINE | 178949-82-1 | 1.29 |
| DISUCCINATE | | |
| AMMONIUM SULFATE | 7783-20-2 | 1.00 |
| SODIUM SULFITE | 7757-83-7 | 0.40 |
| DICETYL PHOSPHATE | 2197-63-9 | 0.37 |
| CETETH-10 PHOSPHATE | 50643-20-4 | 0.29 |
| STEARETH-200 | 9005-00-9 | 0.25 |
| ASCORBIC ACID | 50-81-7 | 0.30 |
| SODIUM HYDROXIDE | 1310-73-2 | 0.13 |
| DISODIUM EDTA | 139-33-3 | 0.09 |
| XANTHAN GUM | 11138-66-2 | 0.04 |
| 2-METHOXYMETHYL-P- | 337906-36-2 | 0.900 |
| PHENYLENEDIAMINE | | |
| RESORCINOL | 108-46-3 | 0.500 |
| N,N-BIS(2-HYDROXYETHYL)-P- | 54381-16-7 | 0.050 |
| PHENYLENEDIAMINE SULFATE | | |
| M-AMINOPHENOL | 591-27-5 | 0.013 |
| 4-AMINO-2-HYDROXYTOLUENE | 2835-95-2 | 0.010 |
| P-AMINOPHENOL | 123-30-8 | 0.009 |
| 1-NAPHTHOL | 90-15-3 | 0.007 |
| PERFUME | | 0.800 |
| WATER up to 100% | | |

### Precursor Composition D—Medium Blonde

| | | Ingredient |
|---|---|---|
| | | Concentration |
| | CAS | in Finished |
| Ingredient INCI Name | Number | Product wt % |
| PROPYLENE GLYCOL | 57-55-6 | 7.63 |
| AMMONIUM HYDROXIDE | 1336-21-6 | 4.94 |
| CETEARYL ALCOHOL | 67762-27-0 | 2.10 |
| TRISODIUM ETHYLENEDIAMINE | 178949-82-1 | 1.29 |
| DISUCCINATE | | |
| AMMONIUM SULFATE | 7783-20-2 | 0.50 |
| SODIUM SULFITE | 7757-83-7 | 0.40 |
| DICETYL PHOSPHATE | 2197-63-9 | 0.37 |
| CETETH-10 PHOSPHATE | 50643-20-4 | 0.29 |
| STEARETH-200 | 9005-00-9 | 0.25 |
| ASCORBIC ACID | 50-81-7 | 0.30 |
| SODIUM HYDROXIDE | 1310-73-2 | 0.13 |
| DISODIUM EDTA | 139-33-3 | 0.09 |
| XANTHAN GUM | 11138-66-2 | 0.04 |
| RESORCINOL | 108-46-3 | 0.360 |
| P-AMINOPHENOL | 123-30-8 | 0.210 |
| M-AMINOPHENOL | 591-27-5 | 0.040 |
| PHENYL METHYL PYRAZOLONE | 89-25-8 | 0.085 |
| P-PHENYLENEDIAMINE | 106-50-3 | 0.016 |
| 4-AMINO-2-HYDROXYTOLUENE | 2835-95-2 | 0.009 |
| PERFUME | | 0.800 |
| WATER Up to 100% | | |

### Precursor Composition D—Medium Brown

| | | Ingredient |
|---|---|---|
| | | Concentration |
| | CAS | in Finished |
| Ingredient INCI Name | Number | Product wt % |
| PROPYLENE GLYCOL | 57-55-6 | 7.63 |
| AMMONIUM HYDROXIDE | 1336-21-6 | 4.30 |
| CETEARYL ALCOHOL | 67762-27-0 | 2.10 |
| TRISODIUM ETHYLENEDIAMINE | 178949-82-1 | 1.29 |
| DISUCCINATE | | |
| AMMONIUM SULFATE | 7783-20-2 | 1.00 |
| SODIUM SULFITE | 7757-83-7 | 0.40 |
| DICETYL PHOSPHATE | 2197-63-9 | 0.37 |
| CETETH-10 PHOSPHATE | 50643-20-4 | 0.29 |
| STEARETH-200 | 9005-00-9 | 0.25 |
| ASCORBIC ACID | 50-81-7 | 0.30 |
| SODIUM HYDROXIDE | 1310-73-2 | 0.13 |
| DISODIUM EDTA | 139-33-3 | 0.09 |
| XANTHAN GUM | 11138-66-2 | 0.04 |
| 2-METHOXYMETHYL-P- | 337906-36-2 | 0.310 |
| PHENYLENEDIAMENE | | |
| RESORCINOL | 108-46-3 | 0.158 |
| 2-METHYLRESORCINOL | 608-25-3 | 0.090 |
| N,N-BIS(2-HYDROXYETHYL)-P- | 54381-16-7 | 0.043 |
| PHENYLENEDIAMINE SULFATE | | |
| M-AMINOPHENOL | 591-27-5 | 0.018 |
| PERFUME | | 0.800 |
| WATER Up to 100% | | |

### Precursor Composition E

| | | Ingredient |
|---|---|---|
| | | Concentration |
| | CAS | in Finished |
| Ingredient INCI Name | Number | Product wt % |
| WATER Up to 100% | — | — |
| HYDROGEN PEROXIDE | 7722-84-1 | 8.90 |
| MINERAL OIL | 8012-95-1; | 7.00 |
| | 8020-83-5; | |
| | 8042-47-5 | |
| CETEARYL ALCOHOL | 67762-27-0 | 2.00 |
| SODIUM CETEARYL SULFATE | 68955-20-4 | 0.90 |
| SALICYLIC ACID | 69-72-7 | 0.10 |
| PHOSPHORIC ACID | 7664-38-2 | 0.08 |
| DISODIUM PHOSPHATE | 7558-79-4 | 0.08 |
| ETIDRONIC ACID | 2809-21-4 | 0.01 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

### Additional Embodiments

The following exemplary embodiments are provided, the numbering of which is not to be construed as designating levels of importance:
Embodiment 1 provides a method for coloring hair comprising the steps of
   i) mixing a developer component with a dye component to obtain a composition A, wherein the developer component comprises an oxidizing agent and the dye component comprises at least one oxidative dye precursor and an alkalising agent and wherein said composition A comprises 0.1 to 10% of oxidising agent (expressed as grams of hydrogen peroxide in 100 grams of the composition A) and 0.1 to 10% alkalizing agent (expressed as grams of ammonium hydroxide in 100 grams of the composition A);
   ii) applying at least a portion of composition A to at least a portion of the front roots of the hair.
   iii) provide another composition B said composition B comprising an oxidising agent concentration (expressed as grams of hydrogen peroxide in 100 grams of the composition B) and an alkalizing agent concentration agent (expressed as grams of ammonium hydroxide in 100 grams of the composition B) such that the ratio of concentration of oxidising agent in composition B to concentration of oxidising agent in composition A is 3:4 or lower, and the ratio of the concentration of alkalizing agent in composition B to the concentration of alkalizing agent in composition A is 4:3 or higher,
   iv) applying at least a portion of the composition B to at least a portions of the back roots of the hair,
   v) rinsing the hair.
Embodiment 2 provides a method according to Embodiment 1 wherein between step iv and v the following steps are also taken:
   v) provide another hair colouring composition C said composition C comprising comprising an oxidizing agent at a concentration wherein the ratio of concentration of oxidizing agent in composition C and the concentration of oxidizing agent in composition A is at least 2:1, and
   vi) applying at least a portion of the composition C to the hair lengths and tips.
Embodiment 3 provides a method according to any preceding Embodiment wherein in step ii) a first portion of composition A is applied to the hair roots at the front and a second portion is not applied to the hair and wherein in step iii) composition B is provided by mixing the second portion of the composition A with a precursor composition D.
Embodiment 4 provides a method according to Embodiment 2 or 3 wherein in step iii) a first portion of composition B is applied to the hair roots at the back and a second portion is not applied to the hair And wherein is step v) composition C is provided by mixing the second portion of composition B with a precursor composition E.
Embodiment 5 provides a method according to any preceding Embodiment wherein said composition A comprises 0.3 to 6%, preferably 1 to 3% of oxidising agent.
Embodiment 6 provides a method according to any preceding Embodiment wherein said composition A comprises 0.3 to 6%, preferably 1 to 3% of alkalizing agent.
Embodiment 7 provides a method according to any preceding Embodiment wherein the ratio of concentration of oxidising agent in composition B to concentration of oxidising agent in composition A is from 1:10 to 3:4, preferably from 1:10 to 1:2, more preferably from 1:10 to 1:3.
Embodiment 8 provides a method according to any preceding Embodiment wherein the ratio of the concentration of alkalizing agent in composition B to the concentration of alkalizing agent in composition A is from 5:4 to 10:1, preferably from 3:2 to 7:1, more preferably from 2:1 to 5:1.
Embodiment 9 provides a method according to Embodiments 2-8 wherein the ratio of concentration of oxidizing agent in composition C and the concentration of oxidizing agent in composition A is at least 3:1, preferably at least 4:1.
Embodiment 10 provides a method according to any preceding Embodiment wherein composition A is applied for a timespan t₁, composition B for a timespan t₂ and composition C (when present) for a timespan t₃ (each timespan being measured in minutes from the end of the application of each composition and the time when rinsing of the hair starts), and wherein t₁>t₂ and when composition C is present t₂>t₃.
Embodiment 11 provides a method according to Embodiment 10 wherein t₁ is from 20 to 60 minutes, t₂ is 2 to 15 minutes shorter than t₂ and t₃, when composition C is present is 2 to 15 minutes shorter than t₂.
Embodiment 12 provides a kit for coloring hair according to the method of any preceding Embodiment said kit comprising the following compositions in separate containers:
   a developer component comprising an oxidising agent,
   a dye component which when mixed with the developer component form composition A,
   a precursor composition D which when mixed with a second portion of composition A forms composition B, and
   optionally a precursor composition E which when mixed with a second portion of composition B forms composition C.
Embodiment 13 provides a kit for coloring hair according to the method of Embodiments 1-11 said kit comprising the following compositions in separate containers:
   a developer component comprising an oxidising agent,
   a dye component which when mixed with the developer component form composition A,
   a precursor composition D which when mixed with a second portion of composition A forms composition B, and
   and optionally a composition C.
Embodiment 14 provides a kit for coloring hair according to the method of Embodiments 1-11 said kit comprising the following compositions in separate containers:
   a developer component comprising an oxidising agent,
   a dye component which when mixed with the developer component form composition A,
   a second developer component, and
   a second dye component which when mixed with the second developer component forms composition B and optionally:
   a third developer component, and
   a third dye component which when mixed with the third developer component forms composition C.
Embodiment 15 provides a kit for coloring hair according to the method of Embodiments 1-11 said kit comprising the following compositions in separate containers:
   a developer component comprising an oxidising agent,
   a dye component which when mixed with the developer component form composition A,
   a second developer component,
   a second dye component which when mixed with the second developer component forms composition B, and optionally:
   and optionally a composition C.
Embodiment 16 provides a method for coloring hair comprising the steps of
   i) mixing a developer component with a dye component to obtain a composition A, wherein the developer component comprises an oxidizing agent and the dye component comprises at least one oxidative dye precursor and an alkalising agent and wherein said composition A comprises 0.1 to 10% of oxidising agent (expressed as grams of hydrogen peroxide in 100 grams of the composition A) and 0.1 to 10% alkalizing agent (expressed as grams of ammonium hydroxide in 100 grams of the composition A);
   ii) applying at least a portion of composition A to at least a portion of the roots of the hair,
   iii) provide another hair colouring composition C said composition C comprising comprising an oxidizing agent at a concentration wherein the ratio of concentration of oxidizing agent in composition C and the concentration of oxidizing agent in composition A is at least 2:1,
   iv) applying at least a portion of the composition C to the hair lengths and tips, and
   v) rinsing the hair.
Embodiment 17 provides a method according to Embodiment 16 wherein in step ii) a first portion of composition A is applied to the hair roots and a second portion is not applied to the hair and wherein in step iii) composition C is provided by mixing the second portion of the composition A with a precursor composition D.
Embodiment 18 provides a method according to any preceding Embodiment wherein said composition A comprises 0.3 to 6%, preferably 1 to 3% of oxidising agent.
Embodiment 19 provides a method according to any preceding Embodiment wherein said composition A comprises 0.3 to 6%, preferably 1 to 3% of alkalizing agent.
Embodiment 20 provides a method according to any preceding Embodiment wherein the ratio of concentration of oxidizing agent in composition C and the concentration of oxidizing agent in composition A is at least 3:1, preferably at least 4:1.
Embodiment 21 provides a method according to any preceding Embodiment wherein composition A is applied for a timespan t₁, and composition C for a timespan t₃ (each timespan being measured in minutes from the end of the application of each composition and the time when rinsing of the hair starts), and wherein t₁>t₃.
Embodiment 22 provides a method according to Embodiment 21 wherein t₁ is from 20 to 60 minutes, and t₃ is 2 to 15 minutes shorter than t₁.
Embodiment 23 provides a kit for coloring hair according to the method of any preceding Embodiment said kit comprising the following compositions in separate containers:
   a developer component comprising an oxidising agent,
   a dye component which when mixed with the developer component forms composition A, and
   a precursor composition E which when mixed with a second portion of composition A forms composition C.
Embodiment 24 provides a kit for coloring hair according to the method of Embodiments 16-23 said kit comprising the following compositions in separate containers:
   a developer component comprising an oxidising agent,
   a dye component which when mixed with the developer component form composition A, and
   a composition C.
Embodiment 25 provides a kit for coloring hair according to the method of Embodiments 16-23 said kit comprising the following compositions in separate containers:
   a developer component comprising an oxidising agent,
   a dye component which when mixed with the developer component form composition A,
   a second developer component, and
   a second dye component which when mixed with the second developer component forms composition C.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. A method for coloring hair comprising the steps of
i) mixing a developer component with a dye component to obtain a composition A, wherein the developer component comprises an oxidizing agent and the dye component comprises at least one oxidative dye precursor and an alkalising agent and wherein said composition A comprises 0.1 to 10% of oxidising agent (expressed as grams of hydrogen peroxide in 100 grams of the composition A) and 0.1 to 10% alkalizing agent (expressed as grams of ammonium hydroxide in 100 grams of the composition A);
ii) applying at least a portion of composition A to at least a portion of the front roots of the hair,
iii) provide another composition B said composition B comprising an oxidising agent concentration (expressed as grams of hydrogen peroxide in 100 grams of the composition B) and an alkalizing agent concentration agent (expressed as grams of ammonium hydroxide in 100 grams of the composition B) such that the ratio of concentration of oxidising agent in composition B to concentration of oxidising agent in composition A is 3:4 or lower, and the ratio of the concentration of alkalizing agent in composition B to the concentration of alkalizing agent in composition A is 4:3 or higher,
iv) applying at least a portion of the composition B to at least a portions of the back roots of the hair,
v) rinsing the hair.

2. A method according to claim 1 wherein between step iv and v the following steps are also taken:
v) provide another hair colouring composition C said composition C comprising comprising an oxidizing agent at a concentration wherein the ratio of concentration of oxidizing agent in composition C and the concentration of oxidizing agent in composition A is at least 2:1, and
vi) applying at least a portion of the composition C to the hair lengths and tips.

3. A method according to claim 1 wherein in step ii) a first portion of composition A is applied to the hair roots at the front and a second portion is not applied to the hair and wherein in step iii) composition B is provided by mixing the second portion of the composition A with a precursor composition D.

4. A method according to claim 2 wherein in step iii) a first portion of composition B is applied to the hair roots at the back and a second portion is not applied to the hair and wherein is step v) composition C is provided by mixing the second portion of composition B with a precursor composition E.

5. A method according to claim 1 wherein said composition A comprises 0.3 to 6% of oxidising agent.

6. A method according to claim 1 wherein said composition A comprises 0.3 to 6% of alkalizing agent.

7. A method according to claim 1 wherein the ratio of concentration of oxidising agent in composition B to concentration of oxidising agent in composition A is from 1:10 to 3:4.

8. A method according to claim 1 wherein the ratio of the concentration of alkalizing agent in composition B to the concentration of alkalizing agent in composition A is from 5:4 to 10:1.

9. A method according to claim 2 wherein the ratio of concentration of oxidizing agent in composition C and the concentration of oxidizing agent in composition A is at least 3:1.

10. A method according to claim 1 wherein composition A is applied for a timespan t₁, composition B for a timespan t₂ and composition C (when present) for a timespan t₃ (each timespan being measured in minutes from the end of the application of each composition and the time when rinsing of the hair starts), and wherein t₁>t₂ and when composition C is present t₂>t₃.

11. A method according to claim 10 wherein t₁ is from 20 to 60 minutes, t₂ is 2 to 15 minutes shorter than t₂ and t₃, when composition C is present is 2 to 15 minutes shorter than t₂.

12. A kit for coloring hair according to the method of claim 1 said kit comprising the following compositions in separate containers:
a developer component comprising an oxidising agent,
a dye component which when mixed with the developer component form composition A,
a precursor composition D which when mixed with a second portion of composition A forms composition B, and
optionally a precursor composition E which when mixed with a second portion of composition B forms composition C.

13. A kit for coloring hair according to the method of claim 1 said kit comprising the following compositions in separate containers:
a developer component comprising an oxidising agent,
a dye component which when mixed with the developer component form composition A,
a precursor composition D which when mixed with a second portion of composition A forms composition B, and
and optionally a composition C.

14. A kit for coloring hair according to the method of claim 1 said kit comprising the following compositions in separate containers:
a developer component comprising an oxidising agent,
a dye component which when mixed with the developer component form composition A,
a second developer component,
a second dye component which when mixed with the second developer component forms composition B and optionally:
a third developer component, and
a third dye component which when mixed with the third developer component forms composition C.

15. A kit for coloring hair according to the method of claim 1 said kit comprising the following compositions in separate containers:
a developer component comprising an oxidising agent,
a dye component which when mixed with the developer component form composition A,
a second developer component, and
a second dye component which when mixed with the second developer component forms composition B and optionally:
and optionally a composition C.

16. A method for coloring hair comprising the steps of
i) mixing a developer component with a dye component to obtain a composition A, wherein the developer component comprises an oxidizing agent and the dye component comprises at least one oxidative dye precursor and an alkalising agent and wherein said composition A comprises 0.1 to 10% of oxidising agent (expressed as grams of hydrogen peroxide in 100 grams of the composition A) and 0.1 to 10% alkalizing agent (expressed as grams of ammonium hydroxide in 100 grams of the composition A);
ii) applying at least a portion of composition A to at least a portion of the roots of the hair,
iii) provide another hair colouring composition C said composition C comprising comprising an oxidizing agent at a concentration wherein the ratio of concentration of oxidizing agent in composition C and the concentration of oxidizing agent in composition A is at least 2:1,
iv) applying at least a portion of the composition C to the hair lengths and tips, and
v) rinsing the hair.

17. A method according to claim 16 wherein in step ii) a first portion of composition A is applied to the hair roots and a second portion is not applied to the hair and wherein in step iii) composition C is provided by mixing the second portion of the composition A with a precursor composition D.

18. A method according to claim 1 wherein said composition A comprises 0.3 to 6% of oxidising agent.

19. A method according to claim 1 wherein said composition A comprises 0.3 to 6% of alkalizing agent.

20. A method according to claim 1 wherein the ratio of concentration of oxidizing agent in composition C and the concentration of oxidizing agent in composition A is at least 3:1.

21. A method according to claim 1 wherein composition A is applied for a timespan t₁, and composition C for a timespan t₃ (each timespan being measured in minutes from the end of the application of each composition and the time when rinsing of the hair starts), and wherein t₁>t₃.

22. A method according to claim 21 wherein t₁ is from 20 to 60 minutes, and t₃ is 2 to 15 minutes shorter than t₁.

23. A kit for coloring hair according to the method of claim 1 said kit comprising the following compositions in separate containers:
a developer component comprising an oxidising agent,
a dye component which when mixed with the developer component forms composition A, and
a precursor composition E which when mixed with a second portion of composition A forms composition C.

24. A kit for coloring hair according to the method of claim 22 said kit comprising the following compositions in separate containers:
a developer component comprising an oxidising agent,
a dye component which when mixed with the developer component form composition A, and
a composition C.

25. A kit for coloring hair according to the method of claim 22 said kit comprising the following compositions in separate containers:
a developer component comprising an oxidising agent,
a dye component which when mixed with the developer component form composition A,
a second developer component, and
a second dye component which when mixed with the second developer component forms composition C.
